(19) United States
(12) Patent Application Publication
(10) Pub. No.: US 2017/0349657 A1
(43) Pub. Date: Dec. 7, 2017

(54) BISPECIFIC ANTIBODIES THAT BIND CD20 AND CD3

(71) Applicant: Xencor, Inc., Monrovia, CA (US)

(72) Inventors: Michael Wayne Saville, San Diego, CA (US); Paul Foster, Rancho Santa Fe, CA (US)

(21) Appl. No.: 15/611,683

(22) Filed: Jun. 1, 2017

Related U.S. Application Data
(60) Provisional application No. 62/344,322, filed on Jun. 1, 2016.
(60) Provisional application No. 62/383,832, filed on Sep. 6, 2016.

(51) Int. Cl. C07K 16/28; A61K 39/395; A61K 39/00

(52) U.S. Cl. CPC C07K 16/2809; A61K 39/395; C07K 16/2887; C07K 2317/35; A61K 2039/505; C07K 2317/73; C07K 2317/92; C07K 2317/21

(57) ABSTRACT

The present invention is directed to methods of administrating bispecific anti-CD20×anti-CD3 antibodies.

### PRIORITY CLAIM

This application claims priority to U.S. Provisional Patent Application No. 62/344,322, filed Jun. 1, 2016 and U.S. Provisional Patent Application No. 62/383,832, filed Sep. 6, 2016 which are expressly incorporated by reference in their entirety.

The instant application contains a Sequence Listing which has been submitted electronically in ASCII format and is hereby incorporated by reference in its entirety. Said ASCII copy, created on May 30, 2017, is named 067461-5193-US ST25.txt and is 71,971 bytes in size.

### BACKGROUND OF THE INVENTION

Antibody-based therapeutics have been used successfully to treat a variety of diseases, including cancer and autoimmune/inflammatory disorders. Yet improvements to this class of drugs are still needed, particularly with respect to enhancing their clinical efficacy. One avenue being explored is the engineering of additional and novel antigen binding sites into antibody-based drugs such that a single immunoglobulin molecule co-engages two different antigens. Because the considerable diversity of the antibody variable region (Fv) makes it possible to produce an Fv that recognizes virtually any molecule, the typical approach to the generation of such bispecific antibodies is the introduction of new variable regions into the antibody.

A number of alternate antibody formats have been explored for bispecific targeting (Chames & Baty, 2009, mAbs 1[6]:1-9; Holliger & Hudson, 2005, Nature Biotechnology 23[9]:1126-1136; Kontermann, mAbs 4(2):182 (2012), all of which are expressly incorporated herein by reference). Initially, bispecific antibodies were made by fusing two cell lines that each produced a single monoclonal antibody (Milstein et al., 1983, Nature 305:537-540). Although the resulting hybrid hybridoma or quadroma did produce bispecific antibodies, they were only a minor population, and extensive purification was required to isolate the desired antibody. An engineering solution to this was the use of antibody fragments to make bispecifics. Because such fragments lack the complex quaternary structure of a full length antibody, variable light and heavy chains can be linked in single genetic constructs. Antibody fragments of many different forms have been generated, including diabodies, single chain diabodies, tandem scFvs, and Fab₂ bispecifics (Chames & Baty, 2009, mAbs 1[6]:1-9; Holliger & Hudson, 2005, Nature Biotechnology 23[9]:1126-1136; expressly incorporated herein by reference). While these formats can be expressed at high levels in bacteria and may have favorable penetration benefits due to their small size, they clear rapidly in vivo and can present manufacturing obstacles related to their production and stability. A principal cause of these drawbacks is that antibody fragments typically lack the constant region of the antibody with its associated functional properties, including larger size, high stability, and binding to various Fc receptors and ligands that maintain long half-life in serum (i.e. the neonatal Fc receptor FcRn) or serve as binding sites for purification (i.e. protein A and protein G).

More recent work has attempted to address the shortcomings of fragment-based bispecifics by engineering dual binding into full length antibody -like formats (Wu et al., 2007, Nature Biotechnology 25[11]:1290-1297; U.S. Ser. No. 12/477,711; Michaelson et al., 2009, mAbs 1[2]:128-141; PCT/U.S.2008/074693; Zuo et al., 2000, Protein Engineering 13[5]:361-367; U.S. Ser. No. 9/865,198; Shen et al., 2006, J Biol Chem 281[16]:10706-10714; Lu et al., 2005, J Biol Chem 280[20]:19665-19672; PCT/U.S.2005/025472; expressly incorporated herein by reference). These formats overcome some of the obstacles of the antibody fragment bispecifics, principally because they contain an Fc region. One significant drawback of these formats is that, because they build new antigen binding sites on top of the homodimeric constant chains, binding to the new antigen is always bivalent.

For many antigens that are attractive as co-targets in a therapeutic bispecific format, the desired binding is monovalent rather than bivalent. For many immune receptors, cellular activation is accomplished by cross-linking of a monovalent binding interaction. The mechanism of cross-linking is typically mediated by antibody/antigen immune complexes, or via effector cell to target cell engagement. For example, the low affinity Fc gamma receptors (FcγRs) such as FcγRIIa, FcγRIIb, and FcγRIIIa bind monovalently to the antibody Fc region. Monovalent binding does not activate cells expressing these FcγRs; however, upon immune complexation or cell-to-cell contact, receptors are cross-linked and clustered on the cell surface, leading to activation. For receptors responsible for mediating cellular killing, for example FcγRIIIa on natural killer (NK) cells, receptor cross-linking and cellular activation occurs when the effector cell engages the target cell in a highly avid format (Bowles & Weiner, 2005, J Immunol Methods 304:88-99, expressly incorporated by reference). Similarly, on B cells the inhibitory receptor FcγRIIb downregulates B cell activation only when it engages into an immune complex with the cell surface B-cell receptor (BCR), a mechanism that is mediated by immune complexation of soluble IgG's with the same antigen that is recognized by the BCR (Heyman 2003, Immunol Lett 88[2]:157-161; Smith and Clatworthy, 2010, Nature Reviews Immunology 10:328-343; expressly incorporated by reference). As another example, CD3 activation of T-cells occurs only when its associated T-cell receptor (TCR) engages antigen-loaded MHC on antigen presenting cells in a highly avid cell-to-cell synapse (Kuhns et al., 2006, Immunity 24:133-139). Indeed nonspecific bivalent cross-linking of CD3 using an anti-CD3 antibody elicits a cytokine storm and toxicity (Perruche et al., 2009, J Immunol 183[2]:953-61; Chatenoud & Bluestone, 2007, Nature Reviews Immunology 7:622-632; expressly incorporated by reference). Thus for practical clinical use, the preferred mode of CD3 co-engagement for redirected killing of targets cells is monovalent binding that results in activation only upon engagement with the co-engaged target.

Accordingly, there is a need for improved bispecific anti-CD-20×anti-CD3 antibodies and the use of such antibodies for use in therapy.

### BRIEF SUMMARY OF THE INVENTION

In one aspect, the present invention provides a method for treating a CD-20 expressing cancer, e.g., a hematologic cancer, e.g., a lymphoma or leukemia, in a human subject, comprising: administering to the human subject having a CD-20 expressing cancer, e.g., a hematologic cancer, e.g., a lymphoma, an intravenous dose of between about 0.1 μg/kg and about 200 μg/kg of a bispecific anti-CD20×anti-CD3 antibody (e.g., XENP13676) once every 6-8 days for a time period sufficient to treat the CD-20 expressing cancer, e.g., the hematologic cancer, e.g., the lymphoma.

In one aspect, provided herein is a bispecific anti-CD20×anti-CD3 antibody (e.g., XENP13676) for use in treating a CD20-expressing cancer, e.g., a hematologic cancer, e.g., a lymphoma or leukemia, in a human subject having a CD20-expressing cancer by administering to the human subject between about 0.1 μg/kg and about 200 μg/kg of an intravenous dose of the bispecific anti-CD20×anti-CD3 antibody (e.g., XENP13676) once every 6-8 days for a time period sufficient to treat the CD20-expressing cancer, e.g., the hematologic cancer, e.g., a lymphoma or leukemia.

In one aspect, the present invention provides a method for treating a CD-20 expressing cancer, e.g., a hematologic cancer, e.g., a lymphoma or leukemia, in a human subject, comprising: administering to the human subject having a CD-20 expressing cancer, e.g., a hematologic cancer, e.g., a lymphoma, an intravenous dose of between about 0.45 μg/kg and about 110 μg/kg of a bispecific anti-CD20×anti-CD3 antibody (e.g., XENP13676) monthly for a time period sufficient to treat the CD20-expressing cancer, e.g., a hematologic cancer, e.g., a lymphoma. In some embodiments, the intravenous dose is between about 28 μg/kg and about 80 μg/kg.

In one aspect, provided herein is a bispecific anti-CD20×anti-CD3 antibody (e.g., XENP13676) for use in treating a CD20-expressing cancer, e.g., a hematologic cancer, e.g., a lymphoma or leukemia, in a human subject having a CD20-expressing cancer by administering to the human subject between about 0.45 μg/kg and about 110 μg/kg of an intravenous dose of the bispecific anti-CD20×anti-CD3 antibody (e.g., XENP13676) monthly for a time period sufficient to treat the CD20-expressing cancer, e.g., the hematologic cancer, e.g., a lymphoma or leukemia. In some embodiments, the intravenous dose is between about 28 μg/kg and about 80 μg/kg. In one aspect, the present invention provides a method for treating a CD-20 expressing cancer, e.g., a hematologic cancer, e.g., a lymphoma or leukemia, in a human subject, comprising: administering to the human subject having a CD-20 expressing cancer, e.g., a hematologic cancer, e.g., a lymphoma, an intravenous dose of between about 0.45 μg/kg and about 110 μg/kg of a bispecific anti-CD20×anti-CD3 antibody (e.g., XENP13676) every other week for a time period sufficient to treat the CD20-expressing cancer, e.g., a hematologic cancer, e.g., a lymphoma. In some embodiments, the intravenous dose is between about 28 μg/kg and about 80 μg/kg.

In one aspect, provided herein is a bispecific anti-CD20×anti-CD3 antibody (e.g., XENP13676) for use in treating a CD20-expressing cancer, e.g., a hematologic cancer, e.g., a lymphoma or leukemia, in a human subject having a CD20-expressing cancer by administering to the human subject between about 0.45 μg/kg and about 110 μg/kg of an intravenous dose of the bispecific anti-CD20×anti-CD3 antibody (e.g., XENP13676) every other week for a time period sufficient to treat the CD20-expressing cancer, e.g., the hematologic cancer, e.g., a lymphoma or leukemia. In some embodiments, the intravenous dose is between about 28 μg/kg and about 80 μg/kg.

In some embodiments, the intravenous dose is between about 0.6 μg/kg and about 0.8 μg/kg; or between about 2.3 μg/kg and about 2.5 μg/kg; or between about 6.5 μg/kg and about 8.5 μg/kg; or between about 18 μg/kg and about 22 μg/kg; or between about 40 μg/kg and about 50 μg/kg; or between about 75 μg/kg and about 85 μg/kg; or between about 120 μg/kg and about 130 μg/kg; or between about 165 μg/kg and about 175 μg/kg.

In one aspect, the methods and antibodies of the present invention are used for treating a lymphoma, wherein said lymphoma is a Non-Hodgkin lymphoma (“NHL”), for example, a B-cell NHL. In some embodiments, the Non-Hodgkin lymphoma is selected from the group consisting of Burkitt's lymphoma (e.g., Endemic Burkitt's Lymphoma and Sporadic Burkitt's Lymphoma), Cutaneous B-Cell Lymphoma, Cutaneous Marginal Zone Lymphoma (MZL), Diffuse Large Cell Lymphoma (DLBCL), Diffuse Mixed Small and Large Cell Lymphoma, Diffuse Small Cleaved Cell, Diffuse Small Lymphocytic Lymphoma, Extranodal Marginal Zone B-cell lymphoma, follicular lymphoma, Follicular Small Cleaved Cell (e.g., Grade 1), Follicular Mixed Small Cleaved and Large Cell (e.g., Grade 2), Follicular Large Cell (e.g., Grade 3), Intravascular Large B-Cell Lymphoma, Intravascular Lymphomatosis, Large Cell Immunoblastic Lymphoma, Large Cell Lymphoma (LCL), Lymphoblastic Lymphoma, MALT Lymphoma, Mantle Cell Lymphoma (MCL), immunoblastic large cell lymphoma, precursor B-lymphoblastic lymphoma, mantle cell lymphoma, chronic lymphocytic leukemia (CLL) (e.g., small lymphocytic lymphoma (SLL)), extranodal marginal zone B-cell lymphoma-mucosa-associated lymphoid tissue (MALT) lymphoma, Mediastinal Large B-Cell Lymphoma, nodal marginal zone B-cell lymphoma, splenic marginal zone B-cell lymphoma, primary mediastinal B-cell lymphoma, lymphoplasmocytic lymphoma, hairy cell leukemia, Waldenstrom's Macroglobulinemia, and primary central nervous system (CNS) lymphoma. In some embodiments, the Non-Hodgkin lymphoma is chronic lymphocytic leukemia (CLL) or small lymphocytic lymphoma (SLL).

In some embodiments, according to the present invention, the intravenous dose is administered to a human subject between about 1 hour and about 3 hours. In some embodiments, according to the present invention, the time period sufficient to treat a CD-20 expressing cancer, e.g., a hematologic cancer, e.g., a lymphoma, in a human subject is between about 3 weeks and 9 weeks.

In some embodiments, the bispecific anti-CD20×anti-CD3 antibody used according to the present invention is XENP13676 as described herein. The XENP13676 antibody includes a first monomer comprising SEQ ID NO: 1, a second monomer comprising SEQ ID NO: 2, and a light chain comprising SEQ ID NO: 3.

In some embodiments, the methods and antibodies of the present invention further comprise, prior to the administering of a bispecific anti-CD20×anti-CD3 antibody (e.g., XENP13676), administering a steroid to the human subject. In some embodiments, the methods of the present invention further comprise, prior to the administering of a bispecific anti-CD20×anti-CD3 antibody, assessing the weight of the human subject.

In some embodiments, the methods and antibodies of the present invention further comprise administering to the human subject another agent selected from an alkylating agent such as bendamustine hydrochloride (e.g., Treanda), chlorambucil (e.g., Leukeran, Ambochlorin, Amboclorin, Linfolizin), cyclophosphamide (e.g., Cytoxan, Clafen, Neosar); a purine analog such as fludarabine phosphate (e.g., Fludara), cladribine (e.g., Leustatin, 2-CdA), pentostatin (Nipent®); an Bcl2 inhibitor such as ABT-737, venetoclax (e.g., Venclexta); a kinase inhibitor such as ibrutinib (e.g., Imbruvica), venetoclax, idelalisib (e.g., Zydelig); an anti-CD52 Ab such as alemtuzumab (Campath®); a corticosteroid such as prednisone, methylprednisolone, or dexamethasone; or CVP (a combination of cyclophosphamide, vincristine, and prednisone), CHOP (a combination of cyclophosphamide, hydroxydaunorubicin, Oncovin® (vincristine), and prednisone) with or without etoposide (e.g., VP-16), a combination of cyclophosphamide and pentostatin, a combination of chlorambucil and prednisone, a combination of fludarabine and cyclophosphamide, or another agent such as mechlorethamine hydrochloride (e.g. Mustargen), doxorubicin (Adriamycin®), methotrexate, oxaliplatin, or cytarabine (ara-C).

In some embodiments, the methods and antibodies of the present invention further comprise administering to the the subject another therapy.

In some embodiments, the therapy is a chemotherapy. In one embodiment, the chemotherapy is selected from the group consisting of : anthracycline (e.g., idarubicin, daunorubicin, doxorubicin (e.g., liposomal doxorubicin)), a anthracenedione derivative (e.g., mitoxantrone), a vinca alkaloid (e.g., vinblastine, vincristine, vindesine, vinorelbine), an alkylating agent (e.g., cyclophosphamide, deacarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, cytarabine, pyrimidine analogs, purine analogs and adenosine deaminase inhibitors (e.g., fludarabine)), an mTOR inhibitor, a proteasome inhibitor (e.g., aclacinomycin A, gliotoxin or bortezomib), an immunomodulator such as thalidomide or a thalidomide derivative (e.g., lenalidomide).

In some embodiments, the therapy is a therapy that ameliorates side effects. In some embodiments, the therapy is selected from the group consisting of: a steroid (e.g., corticosteroid, e.g., methylprednisolone, hydrocortisone), an inhibitor of TNFα, inhibitor of IL-1R, and an inhibitor of IL-6. In some embodiments, the therapy is a combination of a corticosterioid (e.g., methylprednisolone, hydrocortisone) and Benadryl and Tylenol, wherein said corticosterioid, Benadryl and Tylenol are administered to said subject prior to the administration of said anti-CD20 x anti-CD3 antibody (e.g., XENP13676).

### BRIEF DESCRIPTION OF THE DRAWINGS

FIG. 1 depicts a particularly useful bispecific format of the invention, referred to as a “bottle opener”, which is also the format of XENP13676. It should be noted that the scFv and Fab domains can be switched (e.g. anti-CD3 as a Fab, and anti-CD20 as a scFv).

FIG. 2 depicts the sequences of the three polypeptide chains that make up XENP13676, an anti-CD20×anti-CD3 antibody of particular use in the present invention. The CDRs are underlined and the junction between domains is denoted by a slash (“/”). The charged scFv linker is double underlined; as will be appreciated by those in the art, the linker may be substituted with other linkers, and particularly other charged linkers that are depicted in FIG. 7 of U.S. Publication Number 2014/0288275, or other non-charged linkers (SEQ ID NO:441 of U.S. Publication Number 2014/0288275).

FIG. 3A-3E depicts additional anti-CD20×anti-CD3 sequences of the invention, with the CDRs underlined.

FIG. 4A-4D depicts depicts additional bispecific formats of use in the present invention, as are generally described in FIG. 1 and the accompanying Legend and supporting text of U.S. Ser. No. 14/952,714 (incorporated herein by reference).

FIG. 5 is a line graph showing XmAb13676 potently kills multiple CD20-positive B cell lines.

FIG. 6 is a line graph showing XmAb13676 stimulates activation of CD8+ T cells in the presence of CD20-expressing B cell lines.

FIG. 7 is a line graph showing XmAb13676 retains RTCC activity in the presence of rituximab.

FIG. 8 is a line graph showing XmAb13676-mediated CD69 induction on CD8 T cells in CLL and normal PBMC.

FIG. 9 is a bar graph showing CLL depletion in PBMC enriched with normal T cells.

FIG. 10 is a dot graph showing XmAb13676 depletes follicular lymphoma (CD19+ CD10+) cells in patient PBMC samples.

FIG. 11 is a line graph showing XmAb13676 prevents Raji tumor growth in huPBMC-NSG mice.

### DETAILED DESCRIPTION OF THE INVENTION

### I. Definitions

In order that the application may be more completely understood, several definitions are set forth below. Such definitions are meant to encompass grammatical equivalents.

By “CD3” or “cluster of differentiation 3” herein is meant a T-cell co-receptor that helps in activation of both cytooxic T-cell (e.g., CD8+naïve T cells) and T helper cells (e..g, CD4+naïve T cells) and is composed of four distinct chains: one CD3γ chain (e.g., Genbank Accession Numbers NM_000073 and MP_000064 (human)), one CD3δ chain (e.g., Genbank Accession Numbers NM_000732, NM_001040651, NP 00732 and NP 001035741 (human)), and two CD3ε chains (e.g., Genbank Accession Numbers NM 000733 and NP 00724 (human)). The chains of CD3 are highly related cell-surface proteins of the immunoglobulin superfamily containing a single extracellular immunoglobulin domain. The CD3 molecule associates with the T-cell receptor (TCR) and ζ-chain to form the T-cell receptor (TCR) complex, which functions in generating activation signals in T lymphocytes.

By “B-lymphocyte antigen CD20” or “CD20” or “CD20 antigen” or “CD20 Receptor” or “Membrane Spanning 4-Domains A1” or “Membrane-Spanning 4-Domains, Subfamily A, Member 1” or “Leukocyte Surface Antigen Leu-16” or “Bp35” or “B-Lymphocyte Cell-Surface Antigen 1” or “LEU-16” or “CVIDS” or “MS4A1” or “B1” or “S7” herein is meant an activated-glycosylated phosphoprotein expressed on the surface of B-cells and is encoded by the MS4A1 gene in humans (e.g., Genbank Accession Numbers NM_152866, NM_021950, NP_068769 and NP_690605 (human)). CD20 plays a role in the development and differentiation of B-cells into plasma cells.

By “bispecific” or “bispecific antibody” herein is meant any non-native or alternate antibody formats, including those described herein, that engage two different antigens (e.g., CD3×CD20 bispecific antibodies).

By “modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g. the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, -233E or 233E designates an insertion of glutamic acid after position 233 and before position 234. Additionally, -233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233− or E233# or E233( )esignates a deletion of glutamic acid at position 233. Additionally, EDA233− or EDA233190 designates a deletion of the sequence GluAspAla that begins at position 233.

By “variant protein” or “protein variant”, or “variant” as used herein is meant a protein that differs from that of a parent protein by virtue of at least one amino acid modification. Protein variant may refer to the protein itself, a composition comprising the protein, or the amino sequence that encodes it. Preferably, the protein variant has at least one amino acid modification compared to the parent protein, e.g. from about one to about seventy amino acid modifications, and preferably from about one to about five amino acid modifications compared to the parent. As described below, in some embodiments the parent polypeptide, for example an Fc parent polypeptide, is a human wild type sequence, such as the Fc region from IgG1, IgG2, IgG3 or IgG4, although human sequences with variants can also serve as “parent polypeptides”. The protein variant sequence herein will preferably possess at least about 80% identity with a parent protein sequence, and most preferably at least about 90% identity, more preferably at least about 95-98-99% identity. Variant protein can refer to the variant protein itself, compositions comprising the protein variant, or the DNA sequence that encodes it. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain. The Fc variants of the present invention are defined according to the amino acid modifications that compose them. Thus, for example, N434S or 434S is an Fc variant with the substitution serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/4345. It is noted that the order in which substitutions are provided is arbitrary, that is to say that, for example, 428L/4345 is the same Fc variant as M428L/N434S, and so on. For all positions discussed in the present invention that relate to antibodies, unless otherwise noted, amino acid position numbering is according to the EU index. The EU index or EU index as in Kabat or EU numbering scheme refers to the numbering of the EU antibody (Edelman et al., 1969, Proc Natl Acad Sci USA 63:78-85, hereby entirely incorporated by reference.) The modification can be an addition, deletion, or substitution. Substitutions can include naturally occurring amino acids and, in some cases, synthetic amino acids. Examples include U.S. Pat. No. 6,586,207; WO 98/48032; WO 03/073238; U.S.2004-0214988A1; WO 05/35727A2; WO 05/74524A2; J. W. Chin et al., (2002), Journal of the American Chemical Society 124:9026-9027; J. W. Chin, & P. G. Schultz, (2002), ChemBioChem 11:1135-1137; J. W. Chin, et al., (2002), PICAS United States of America 99:11020-11024; and, L. Wang, & P. G. Schultz, (2002), Chem. 1-10, all entirely incorporated by reference.

As used herein, “protein” herein is meant at least two covalently attached amino acids, which includes proteins, polypeptides, oligopeptides and peptides. The peptidyl group may comprise naturally occurring amino acids and peptide bonds, or synthetic peptidomimetic structures, i.e. “analogs”, such as peptoids (see Simon et al., PNAS USA 89(20):9367 (1992), entirely incorporated by reference). The amino acids may either be naturally occurring or synthetic (e.g. not an amino acid that is coded for by DNA); as will be appreciated by those in the art. For example, homo-phenylalanine, citrulline, ornithine and noreleucine are considered synthetic amino acids for the purposes of the invention, and both D- and L-(R or S) configured amino acids may be utilized. The variants of the present invention may comprise modifications that include the use of synthetic amino acids incorporated using, for example, the technologies developed by Schultz and colleagues, including but not limited to methods described by Cropp & Shultz, 2004, Trends Genet. 20(12):625-30, Anderson et al., 2004, Proc Natl Acad Sci USA 101 (2):7566-71, Zhang et al., 2003, 303(5656):371-3, and Chin et al., 2003, Science 301(5635):964-7, all entirely incorporated by reference. In addition, polypeptides may include synthetic derivatization of one or more side chains or termini, glycosylation, PEGylation, circular permutation, cyclization, linkers to other molecules, fusion to proteins or protein domains, and addition of peptide tags or labels.

By “residue” as used herein is meant a position in a protein and its associated amino acid identity. For example, Asparagine 297 (also referred to as Asn297 or N297) is a residue at position 297 in the human antibody IgG1.

By “Fab” or “Fab region” as used herein is meant the polypeptide that comprises the VH, CH1, VL, and CL immunoglobulin domains. Fab may refer to this region in isolation, or this region in the context of a full length antibody, antibody fragment or Fab fusion protein. By “Fv” or “Fv fragment” or “Fv region” as used herein is meant a polypeptide that comprises the VL and VH domains of a single antibody. As will be appreciated by those in the art, these generally are made up of two chains.

By “amino acid” and “amino acid identity” as used herein is meant one of the 20 naturally occurring amino acids that are coded for by DNA and RNA.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor”, “FcγR” or “FcqammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or Fc665 R isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants can be used to increase binding to the FcRn receptor, and in some cases, to increase serum half-life.

By “parent polypeptide” as used herein is meant a starting polypeptide that is subsequently modified to generate a variant. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below.

By “Fc” or “Fc region” or “Fc domain” as used herein is meant the polypeptide comprising the constant region of an antibody excluding the first constant region immunoglobulin domain and in some cases, part of the hinge. Thus Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). Although the boundaries of the Fc region may vary, the human IgG heavy chain Fc region is usually defined to include residues C226 or P230 to its carboxyl-terminus, wherein the numbering is according to the EU index as in Kabat. In some embodiments, as is more fully described below, amino acid modifications are made to the Fc region, for example to alter binding to one or more FcγR receptors or to the FcRn receptor.

By “heavy constant region” herein is meant the CH1-hinge-CH2-CH3 portion of an antibody.

By “position” as used herein is meant a location in the sequence of a protein. Positions may be numbered sequentially, or according to an established format, for example the EU index for antibody numbering.

By “target antigen” as used herein is meant the molecule that is bound specifically by the variable region of a given antibody. The two target antigens of the present invention are human CD3 and human CD20.

By “strandedness” in the context of the monomers of the heterodimeric antibodies of the invention herein is meant that, similar to the two strands of DNA that “match”, heterodimerization variants are incorporated into each monomer so as to preserve the ability to “match” to form heterodimers. For example, if some pI variants are engineered into monomer A (e.g. making the pI higher) then steric variants that are “charge pairs” that can be utilized as well do not interfere with the pI variants, e.g. the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer that incorporates one set of the pair will go, such that pI separation is maximized using the pI of the skews as well.

By “target cell” as used herein is meant a cell that expresses a target antigen.

By “variable region” as used herein is meant the region of an immunoglobulin that comprises one or more Ig domains substantially encoded by any of the Vκ, Vλ, and/or VH genes that make up the kappa, lambda, and heavy chain immunoglobulin genetic loci respectively.

By “wild type or WT” herein is meant an amino acid sequence or a nucleotide sequence that is found in nature, including allelic variations. A WT protein has an amino acid sequence or a nucleotide sequence that has not been intentionally modified.

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogeneous host cells.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10-4 M, at least about 10-5 M, at least about 10-6 M, at least about 10-7 M, at least about 10-8 M, at least about 10-9 M, alternatively at least about 10-10 M, at least about 10-11 M, at least about 10-12 M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore assay.

As used herein, the term “target activity” refers to a biological activity capable of being modulated by a selective modulator. Certain exemplary target activities include, but are not limited to, binding affinity, signal transduction, enzymatic activity, tumor growth, effects on particular biomarkers related to CD20 disorder pathology.

By “refractory” in the context of a cancer is intended the particular cancer is resistant to, or non-responsive to, therapy with a particular therapeutic agent. A cancer can be refractory to therapy with a particular therapeutic agent either from the onset of treatment with the particular therapeutic agent (i.e., non-responsive to initial exposure to the therapeutic agent), or as a result of developing resistance to the therapeutic agent, either over the course of a first treatment period with the therapeutic agent or during a subsequent treatment period with the therapeutic agent.

As used herein, the IC₅₀ refers to an amount, concentration or dosage of a particular test compound that achieves a 50% inhibition of a maximal response, such as inhibition of the biological activity of CD20, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

### II. Overview

The invention provides methods of treating a cancer that include cells expressing CD20 (“CD20-expressing cancer”), for example, a hematologic cancer, such as lymphoma or leukemia through the administration of certain bispecific anti-CD20×anti-CD3 antibodies (e.g., XENP13676) at particular dosages. The present invention also provides methods of combination therapies, for example, methods of treating a cancer that include cells expressing CD20 (“CD20-expressing cancer”), e.g., a hematologic cancer, such as lymphoma or leukemia, through the administration of certain bispecific anti-CD20×anti-CD3 antibodies (e.g., XENP13676) in combination with one or more chemotherapies or therapies that can ameliorate side effects of an anti-CD20×anti-CD3 antibody.

### III. Antibodies

The present invention is directed to the administration of bispecific anti-CD20 x anti-CD3 antibodies (e.g., XENP13676) for the treatment of particular lymphomas as outlined herein, as outlined in U.S. Ser. Nos. 14/952,714, 15/141,350, and 62/085,027, all of which are expressly incorporated herein by reference, particularly for the bispecific formats of the figures, as well as all sequences, Figures and accompanying Legends therein.

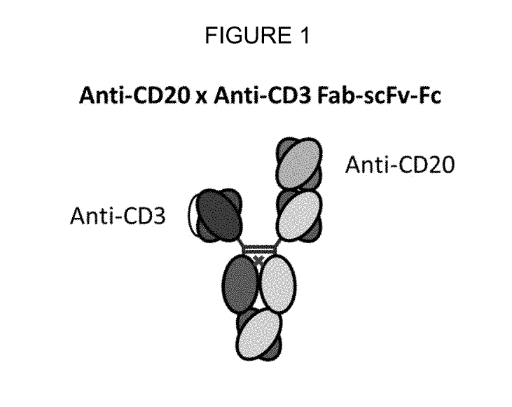

In some embodiments, the bispecific anti-CD20×anti-CD3 antibodies (e.g., XENP13676) have a “bottle opener” format as is generally depicted in FIG. 1. In this embodiment, the anti-CD3 antigen binding domain is the scFv-Fc domain monomer and the anti-CD20 antigen binding domain is the Fab monomer (terms as used in U.S. Publication Nos. 2014/0288275 and 2014-0294823 as well as in U.S. Ser. No. 15/141,350, all of which are expressly incorporated by reference in their entirety and specifically for all the definitions, sequences of anti-CD3 antigen binding domains and sequences of anti-CD20 antigen binding domains).

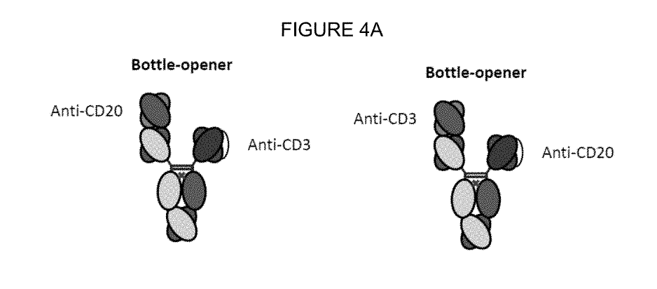

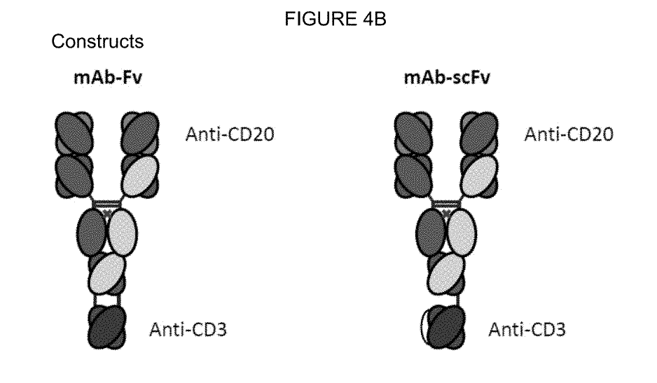

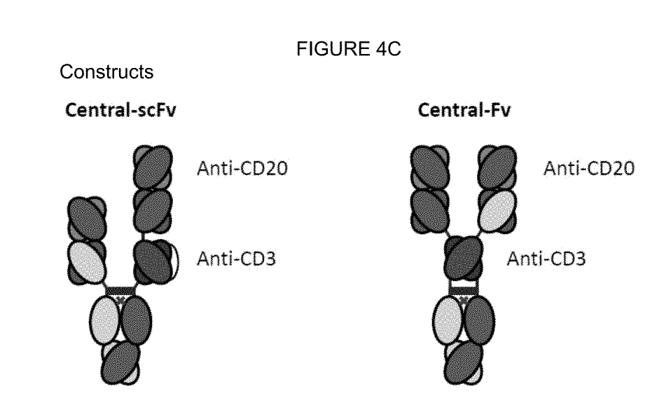

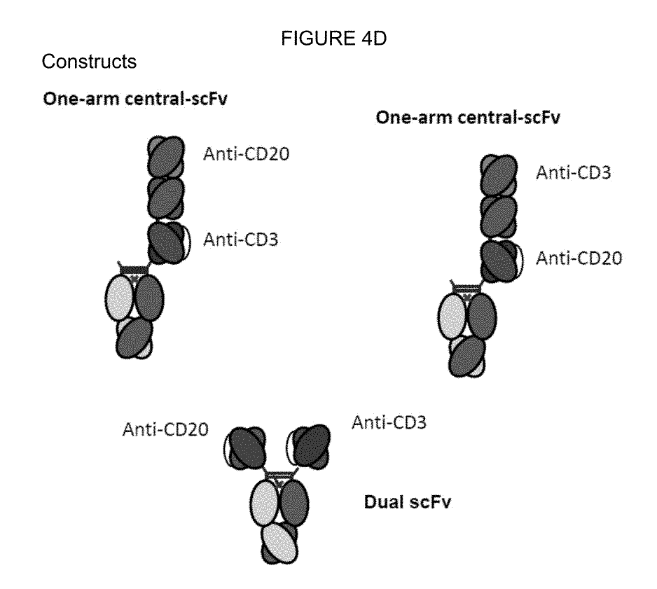

Alternate formats for the bispecific, heterodimeric anti-CD20×anti-CD3 antibodies of the invention are shown in FIG. 4, which also generally rely on the use of Fabs and scFv domains in different formats.

In addition, it is also possible to make non-heterodimeric anti-CD20×anti-CD3 bispecific antibodies as are known in the art, that can be dosed at the same dosage levels as described herein for the heterodimeric bispecific anti-CD20×anti-CD3 antibodies.

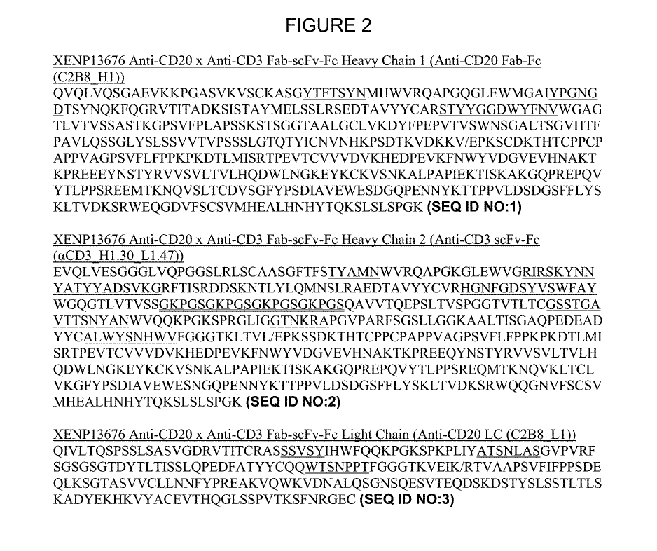

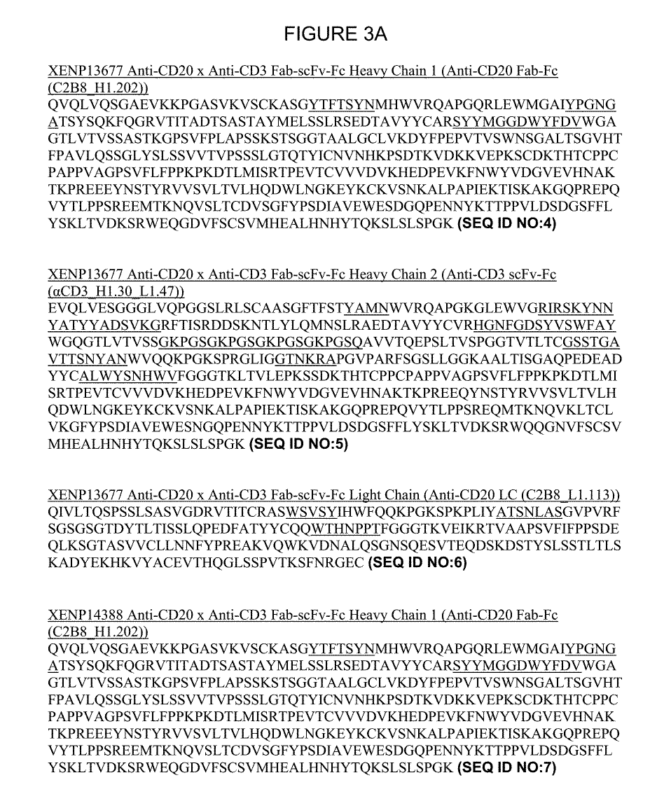

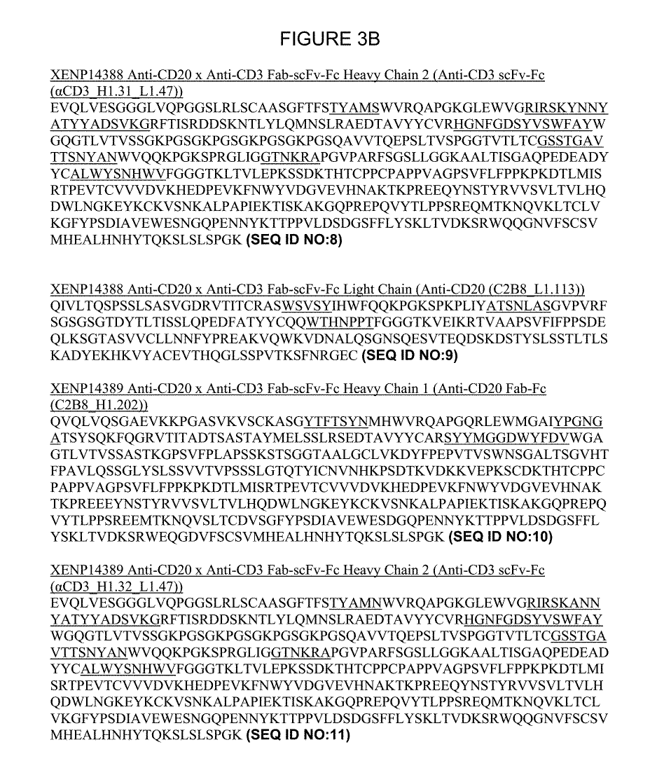

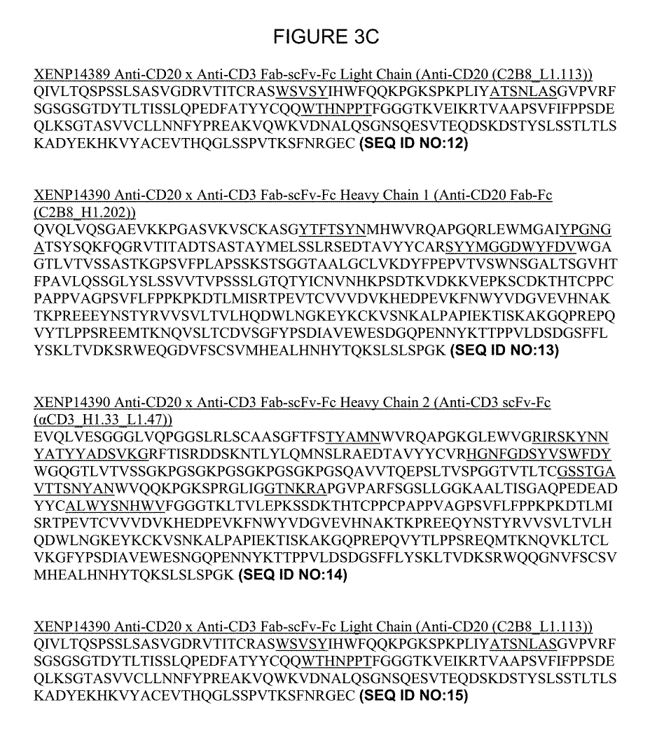

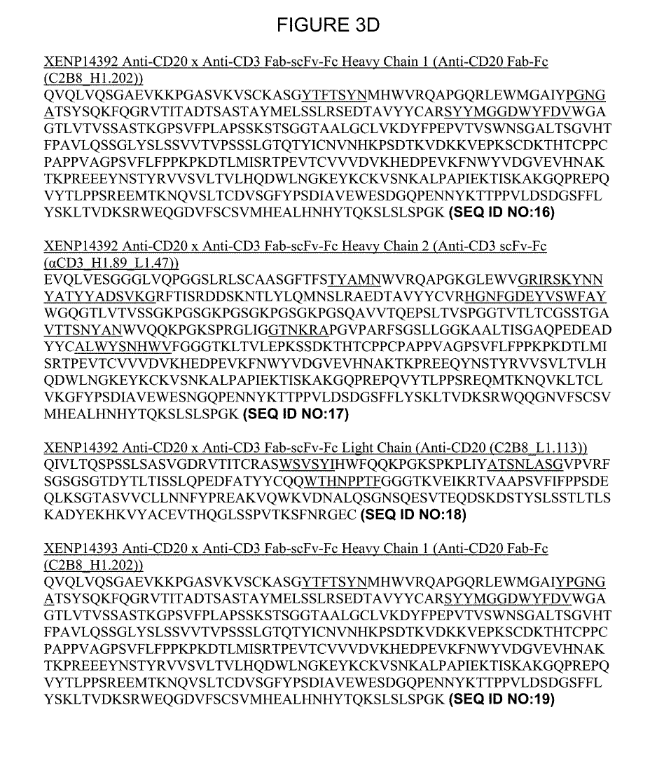

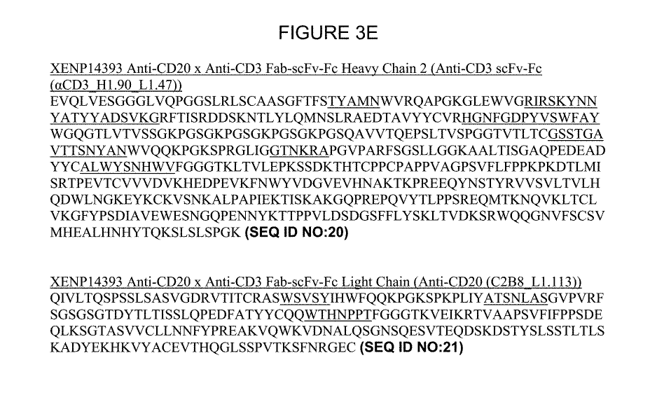

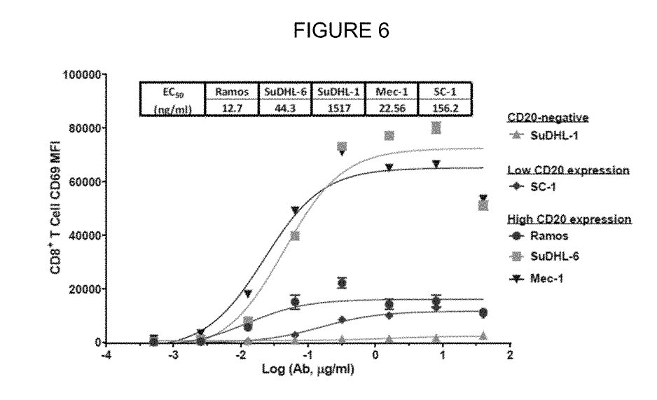

The anti-CD3 scFv antigen binding domain can have the sequence depicted in FIG. 2, or can be selected from:
1) the set of 6 CDRs (vhCDR1, vhCDR2, vhCDR3, v1CDR1, v1CDR2 and v1CDR3) from any anti-CD3 antigen binding domain sequence depicted in FIGS. 2 and 6 of U.S. Publication No. 2014/0288275;
2) the variable heavy and variable light chains from any anti-CD3 antigen binding domain sequence depicted in FIGS. 2 and 6 of U.S. Publication No. 2014/0288275;
3) the scFv domains from any anti-CD3 scFv sequence depicted in FIG. 2 of U.S. Publication No. 2014/0288275;
4) any of the anti-CD3 antigen binding domains of FIGS. 2, 3, 4, 5, 6, and 7 of U.S. Ser. No. 14/952,714;
5) other anti-CD3 variable heavy and variable light chains as are known in the art, that can be combined to form scFvs (or Fabs, when the format is reversed or an alternative format is used); and
6) any of the anti-CD3 antigen binding domains of FIGS. 2, 3, 4, 5, 6, and 7 of U.S. Ser. No. 14/952,714.

The anti-CD20 Fab binding domain can have the sequence depicted in FIG. 2 or 4, or can be selected from:
1) The set of 6 CDRs (vhCDR1, vhCDR2, vhCDR3, v1CDR1, v1CDR2 and v1CDR3) from any anti-CD20 antigen binding domain sequence depicted in U.S. Ser. No. 62/084,908;
2) The variable heavy and variable light chains from any anti-CD20 antigen binding domain sequence depicted in U.S. Ser. No. 62/084,908, including those depicted in FIGS. 2, 3 and 12; and
3) Other anti-CD20 variable heavy and variable light chains as are known in the art, that can be combined to form Fabs (or scFvs, when the format is reversed or an alternative format is used).

One bispecific antibody of particular use in the present invention, XENP13676, is shown in FIG. 2. The XENP13676 antibody includes a first monomer comprising SEQ ID NO: 1, a second monomer comprising SEQ ID NO: 2, and a light chain comprising SEQ ID NO: 3.

The bispecific anti-CD20×anti-CD3 antibodies (e.g., XENP13676) of the invention are made as is known in the art. The invention further provides nucleic acid compositions encoding the bispecific anti-CD20×anti-CD3 antibodies (e.g., XENP13676) of the invention. As will be appreciated by those in the art, the nucleic acid compositions will depend on the format and scaffold of the bispecific anti-CD20×anti-CD3 antibodies (e.g., XENP13676). Thus, for example, when the format requires three amino acid sequences, such as for the triple F format (e.g. a first amino acid monomer comprising an Fc domain and a scFv, a second amino acid monomer comprising a heavy chain and a light chain), three nucleic acid sequences can be incorporated into one or more expression vectors for expression. Similarly, some formats (e.g. dual scFv formats such as disclosed in FIG. 4) only two nucleic acids are needed; again, they can be put into one or two expression vectors.

As is known in the art, the nucleic acids encoding the components of the invention can be incorporated into expression vectors as is known in the art, and depending on the host cells used to produce the bispecific anti-CD20×anti-CD3 antibodies of the invention (e.g., XENP13676). Generally the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors of the invention are then transformed into any number of different types of host cells as is well known in the art, including mammalian, bacterial, yeast, insect and/or fungal cells, with mammalian cells (e.g. CHO cells), finding use in many embodiments.

In some embodiments, nucleic acids encoding each monomer and the optional nucleic acid encoding a light chain, as applicable depending on the format, are each contained within a single expression vector, generally under different or the same promoter controls. In embodiments of particular use in the present invention, each of these two or three nucleic acids are contained on a different expression vector.

The heterodimeric bispecific anti-CD20×anti-CD3 antibodies of the invention (e.g., XENP13676) are made by culturing host cells comprising the expression vector(s) as is well known in the art. Once produced, traditional antibody purification steps are done, including an ion exchange chromatography step. As discussed in U.S. Ser. No. 14/205,248 and WO2014/145806, hereby incorporated by reference in their entirety and particularly for the discussions concerning purification, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI) of each monomer so that such that each monomer has a different pI and the heterodimer also has a distinct pI, thus facilitating isoelectric purification of the “triple F” heterodimer (e.g., anionic exchange columns, cationic exchange columns). These substitutions also aid in the determination and monitoring of any contaminating dual scFv-Fc and mAb homodimers post-purification (e.g., IEF gels, cIEF, and analytical IEX columns).

Once made, the bispecific anti-CD20×anti-CD3 antibodies (e.g., XENP13676) are administered to patients in dosages as outlined herein.

### IV. Pharmaceutical Compositions and Pharmaceutical Administration

The bispecific anti-CD20×anti-CD3 antibodies of the invention (e.g., XENP13676) can be incorporated into pharmaceutical compositions suitable for administration to a subject for the methods described herein, e.g., weekly, intravenous dosing. Typically, the pharmaceutical composition comprises a bispecific anti-CD20×anti-CD3 antibody of the invention (e.g., XENP13676) and a pharmaceutically acceptable carrier. As used herein, “pharmaceutically acceptable carrier” includes any and all solvents, dispersion media, coatings, isotonic and absorption delaying agents, and the like that are physiologically compatible and are suitable for administration to a subject for the methods described herein. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as surfactants (such as nonionic surfactants) wetting or emulsifying agents, preservatives or buffers (such as an organic acid, which as a citrate), which enhance the shelf life or effectiveness of the bispecific anti-CD20×anti-CD3 antibody (e.g., XENP13676). An example of pharmaceutically acceptable carriers include polysorbates (polysorbate-80).

The pharmaceutical compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form depends on the intended mode of administration and therapeutic application. Exemplary compositions are in the form of injectable or infusible solutions, such as compositions similar to those used for passive immunization of humans with other antibodies. In an exemplary embodiment, the mode of administration is intravenous. In an exemplary embodiment, the antibody is administered by intravenous infusion or injection.

Pharmaceutical compositions typically must be sterile and stable under the conditions of manufacture and storage. The pharmaceutical composition can be formulated as a solution, microemulsion, dispersion, liposome, or other ordered structure suitable to high drug concentration. Sterile injectable solutions can be prepared by incorporating the antibody in the required amount in an appropriate solvent with one or a combination of ingredients enumerated herein, as required, followed by filtered sterilization. Generally, dispersions are prepared by incorporating the antibody into a sterile vehicle that contains a basic dispersion medium and the required other ingredients from those enumerated herein. In the case of sterile powders for the preparation of sterile injectable solutions, in an exemplary embodiment, the method of preparation is vacuum drying and freeze-drying that yields a powder of the antibody plus any additional desired carrier from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The bispecific anti-CD20×anti-CD3 antibodies of the present invention (e.g., XENP13676) can be administered by a variety of methods known in the art. In an exemplary embodiment, the route/mode of administration is intravenous injection. As will be appreciated by the skilled artisan, the route and/or mode of administration will vary depending upon the desired results. In certain embodiments, the bispecific anti-CD20×anti-CD3 antibody (e.g., XENP13676) may be prepared with a carrier that will protect the antibody against rapid release, such as a controlled release formulation, including implants, transdermal patches, and microencapsulated delivery systems. Biodegradable, biocompatible polymers can be used, such as ethylene vinyl acetate, polyethylene glycol (PEG), polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

### V. Methods of Treatment

In an exemplary embodiment, the invention provides methods for treating CD20+B cell malignancies, including, but not limited to, B-cell non-hodgkins lymphoma, chronic lymphocytic leukemia, small lymphocytic leukemia, B-cell prolymphoctyic leukemia, transformed leukemia, Burkitt's lymphoma, Mantle cell lymphoma, hairy cell leukemia, splenic marginal zone lympoma, Waldenstrom's Macroglobulinemia, variant hairy cell leukemia, splenic B cell lymphoma/leukemia, lymphoplasmacytic lymphoma, extranodal marginal zone lymphoma of mucosa associated lymphoid tissue, MALT lymphoma, nodal marginal zone B cell lymphoma, follicular lympohma, in situ follicular neoplasia, duodenal-type follicular lymphoma, large B cell lymphoma with IFR4 rearrangement, primary cutaneous follicle center lymphoma, diffuse large B-cell lymphoma (DLBCL), T-cell/histocyte-rich large B cell lymphoma, primary cutaneous DLBCL, leg type, EBV-positive DLBCL, NOS, EBV-positive mucocutaneous ulcer, DLCBL associated with chronic inflammation, lyphomatoid granulomatosis, primary mediastinal (thymic) large B cell lymphoma, intravascular large B-cell lymphoma, ALK+large B-cell lymphoma, plasmablastic lympohma, primary effusion lymphoma, HHV8+DLBCL, Burkitt-like lymphoma with llq aberration, high grade B cell lymphoma, B-cell lypmphoma, unclassifiable, post-transplant lymphoproliferation disorder, and PTLD.

### VI. Methods of Treating Lymphoma

In an exemplary embodiment, the invention provides a method for treating lymphoma in a human subject, comprising administering to the human subject having lymphoma an amount of a bispecific anti-CD20×anti-CD3 antibody described herein (e.g., XENP13676) in a dosage regimen described herein for a time period sufficient to treat the lymphoma. In an exemplary embodiment, the lymphoma is not a Hodgkin's lymphoma. In an exemplary embodiment, the lymphoma is a non-Hodgkin's lymphoma.

### a) Non Hodgkin's Lymphoma

Non-Hodgkin lymphomas (NHL) are a diverse group of malignancies that are predominately of B-cell origin. NHL may develop in any organs associated with lymphatic system such as spleen, lymph nodes or tonsils and can occur at any age. NHL is often marked by enlarged lymph nodes, fever, and weight loss. NHL is classified as either B-cell or T-cell NHL. Lymphomas related to lymphoproliferative disorders following bone marrow or stem cell transplantation are usually B-cell NHL. NHL has been divided into low-, intermediate-, and high-grade categories by virtue of their natural histories (see “The Non-Hodgkin's Lymphoma Pathologic Classification Project,” Cancer 49 (1982):2112-2135). The low-grade lymphomas are indolent, with a median survival of 5 to 10 years (Horning and Rosenberg (1984) N. Engl. J. Med. 311:1471-1475). Although chemotherapy can induce remissions in the majority of indolent lymphomas, cures are rare and most patients eventually relapse, requiring further therapy. The intermediate- and high-grade lymphomas are more aggressive tumors, but they have a greater chance for cure with chemotherapy. However, a significant proportion of these patients will relapse and require further treatment.

In an exemplary embodiment, the lymphoma is a Non-Hodgkin's lymphoma (NHL). In another exemplary embodiment, the lymphoma is a B-cell NHL. In another exemplary embodiment, the lymphoma is selected from the group consisting of Burkitt's lymphoma (e.g., Endemic Burkitt's Lymphoma and Sporadic Burkitt's Lymphoma), Cutaneous B-Cell Lymphoma, Cutaneous Marginal Zone Lymphoma (MZL), Diffuse Large Cell Lymphoma (DLBCL), Diffuse

Mixed Small and Large Cell Lymphoma, Diffuse Small Cleaved Cell, Diffuse Small Lymphocytic Lymphoma, Extranodal Marginal Zone B-cell lymphoma, follicular lymphoma, Follicular Small Cleaved Cell (Grade 1), Follicular Mixed Small Cleaved and Large Cell (Grade 2), Follicular Large Cell (Grade 3), Intravascular Large B-Cell Lymphoma, Intravascular Lymphomatosis, Large Cell Immunoblastic Lymphoma, Large Cell Lymphoma (LCL), Lymphoblastic Lymphoma, MALT Lymphoma, Mantle Cell Lymphoma (MCL), immunoblastic large cell lymphoma, precursor B-lymphoblastic lymphoma, mantle cell lymphoma, chronic lymphocytic leukemia (CLL)/small lymphocytic lymphoma (SLL), extranodal marginal zone B-cell lymphoma-mucosa-associated lymphoid tissue (MALT) lymphoma, Mediastinal Large B-Cell Lymphoma, nodal marginal zone B-cell lymphoma, splenic marginal zone B-cell lymphoma, primary mediastinal B-cell lymphoma, lymphoplasmocytic lymphoma, hairy cell leukemia, Waldenstrom's Macroglobulinemia, and primary central nervous system (CNS) lymphoma.

In an exemplary embodiment, the disease is selected from the group consisting of low-grade and/or follicular NHL, diffuse large B cell lymphoma, Burkitt's or other high-grade NHL, mantle cell lymphoma, MALT lymphoma, Waldenstrom's macroglobulinemia.

Further disclosed herein, in certain embodiments, is a method for treating relapsed or refractory non-Hodgkin's lymphoma in a human subject in need thereof, comprising: administering to the human subject a therapeutically effective amount of a bispecific anti-CD20×anti-CD3 antibody described herein (e.g., XENP13676). In some embodiments, the non-Hodgkin's lymphoma is relapsed or refractory diffuse large B-cell lymphoma (DLBCL), relapsed or refractory mantle cell lymphoma, or relapsed or refractory follicular lymphoma.

### b) CLL/SLL

Chronic lymphocytic leukemia and small lymphocytic lymphoma (CLL/SLL) are commonly thought as the same disease with slightly different manifestations. Where the cancerous cells gather determines whether it is called CLL or SLL. When the cancer cells are primarily found in the lymph nodes, lima bean shaped structures of the lymphatic system (a system primarily of tiny vessels found in the body), it is called SLL. SLL accounts for about 5% to 10% of all lymphomas. When most of the cancer cells are in the bloodstream and the bone marrow, it is called CLL. In an exemplary embodiment, the disease is Richter's syndrome.

Both CLL and SLL are slow-growing diseases, although CLL, which is much more common, tends to grow slower. CLL and SLL are treated the same way. They are usually not considered curable with standard treatments, but depending on the stage and growth rate of the disease, most patients live longer than 10 years. Occasionally over time, these slow-growing lymphomas may transform into a more aggressive type of lymphoma.

Chronic lymphoid leukemia (CLL) is the most common type of leukemia. It is estimated that 100,760 people in the United States are living with or are in remission from CLL. Most (>75%) people newly diagnosed with CLL are over the age of 50. Currently CLL treatment focuses on controlling the disease and its symptoms rather than on an outright cure. CLL is treated by chemotherapy, radiation therapy, biological therapy, or bone marrow transplantation. Symptoms are sometimes treated surgically (splenectomy removal of enlarged spleen) or by radiation therapy (“de-bulking” swollen lymph nodes). Though CLL progresses slowly in most cases, it is considered generally incurable. Certain CLLs are classified as high-risk. As used herein, “high risk CLL” means CLL characterized by at least one of the following 1) 17p13-; 2) 11q22-; 3) unmutated IgVH together with ZAP-70+ and/or CD38+; or 4) trisomy 12.

CLL treatment is typically administered when the patient's clinical symptoms or blood counts indicate that the disease has progressed to a point where it may affect the patient's quality of life.

Small lymphocytic leukemia (SLL) is very similar to CLL described supra, and is also a cancer of B-cells. In SLL the abnormal lymphocytes mainly affect the lymph nodes. However, in CLL the abnormal cells mainly affect the blood and the bone marrow. The spleen may be affected in both conditions. SLL accounts for about 1 in 25 of all cases of non-Hodgkin lymphoma. It can occur at any time from young adulthood to old age, but is rare under the age of 50. SLL is considered an indolent lymphoma. This means that the disease progresses very slowly, and patients tend to live many years after diagnosis. However, most patients are diagnosed with advanced disease, and although SLL responds well to a variety of chemotherapy drugs, it is generally considered to be incurable. Although some cancers tend to occur more often in one gender or the other, cases and deaths due to SLL are evenly split between men and women. The average age at the time of diagnosis is 60 years.

Although SLL is indolent, it is persistently progressive. The usual pattern of this disease is one of high response rates to radiation therapy and/or chemotherapy, with a period of disease remission. This is followed months or years later by an inevitable relapse. Re-treatment leads to a response again, but again the disease will relapse. This means that although the short-term prognosis of SLL is quite good, over time, many patients develop fatal complications of recurrent disease. Considering the age of the individuals typically diagnosed with CLL and SLL, there is a need in the art for a simple and effective treatment of the disease with minimum side-effects that do not impede on the patient's quality of life. The instant invention fulfills this long standing need in the art.

In an exemplary embodiment, the invention provides a method for treating CLL in a human subject, comprising administering to the human subject having CLL an amount of a bispecific anti-CD20×anti-CD3 antibody described herein (e.g., XENP13676) in a dossage regimen described herein for a time period sufficient to treat the CLL.

In an exemplary embodiment, the invention provides a method for treating SLL in a human subject, comprising administering to the human subject having SLL an amount of a bispecific anti-CD20×anti-CD3 antibody described herein (e.g., XENP13676) in a dossage regimen described herein for a time period sufficient to treat the SLL.

### VII. Patient Selection

Patient can be selected based on CD20 expression level in a sample (e.g., a tissue sample or a blood sample) obtained from the patient. CD20 expression level can be determined by an assay known in the art, e.g., flow cytometry, immunohistochemistry, Western blotting, immunofluorescent assay, radioimmunoassay (MA), enzyme-linked immunosorbent assay (ELISA), homogeneous time resolved fluorescence (HTRF), positron emission tomography (PET), or any other immune detection with an antibody or antibody fragment against CD20 protein.

Blood samples can be collected from a patient using any method known in the art, e.g., by venipuncture or fingerstick. Particular types of blood cells can be isolated, expanded, frozen, and used at a later time. Tissue samples can be obtained from a patient using any method known in the art, e.g., by biopsy or surgery. CT imaging, ultrasound, or an endoscope can be used to guide this type of procedure. The sample may be flash frozen and stored at −80° C. for later use. The sample may also be fixed with a fixative, such as formaldehyde, paraformaldehyde, or acetic acid/ethanol. RNA or protein may be extracted from a fresh, frozen or fixed sample for analysis.

### VIII. Dosage Regimen

In some embodiments, the bispecific anti-CD20×anti-CD3 antibody (e.g., XENP13676) is administered according to a dosage regimen described herein. Dosage regimens are adjusted to provide the optimum desired response (e.g., a therapeutic response). The efficient dosages and the dosage regimens for the bispecific anti-CD20×anti-CD3 antibodies used in the present invention (e.g., XENP13676) depend on the disease or condition to be treated and may be determined by the persons skilled in the art.

In an exemplary embodiment, the bispecific anti-CD20×anti-CD3 antibody (e.g., XENP13676) is administered by infusion once every 6-8 days in an amount of from about 0.1 μg/kg and about 125 μg/kg.

In an exemplary embodiment, the bispecific anti-CD20×anti-CD3 antibody (e.g., XENP13676) is administered intravenously by infusion monthly in an amount of from about 0.7 μg/kg to about 170 μg/kg, e.g., about 2.4 μg/kg to about 170 μg/kg, about 7.5 μg/kg to about 170 μg/kg, about 20 μg/kg to about 170 μg/kg, about 45 μg/kg to about 170 μg/kg, about 80 μg/kg to about 170 μg/kg, about 125 μg/kg to about 170 μg/kg, about 0.7 μg/kg to about 125 μg/kg, about 2.4 μg/kg to about 125 μg/kg, about 7.5 μg/kg to about 125 μg/kg, about 20 μg/kg to about 125 μg/kg, about 45 μg/kg to about 125 μg/kg, about 80 μg/kg to about 125 μg/kg, about 0.7 μg/kg to about 80 μg/kg, about 2.4 μg/kg to about 80 μg/kg, about 7.5 μg/kg to about 80 μg/kg, about 20 μg/kg to about 80 μg/kg, about 45 μg/kg to about 80 μg/kg, about 0.7 μg/kg to about 45 μg/kg, about 2.4 μg/kg to about 45 μg/kg, about 7.5 μg/kg to about 45 μg/kg, about 20 μg/kg to about 45 μg/kg, about 0.7 μg/kg to about 20 μg/kg, about 2.4 μg/kg to about 20 μg/kg, about 7.5 μg/kg to about 20 μg/kg, about 0.7 μg/kg to about 7.5 μg/kg, about 2.4 μg/kg to about 7.5 μg/kg, about 0.7 μg/kg to about 2.4 μg/kg. In an exemplary embodiment, the bispecific anti-CD20×anti-CD3 antibody (e.g., XENP13676) is administered intravenously by infusion monthly in an amount of from about 0.6 μg/kg and about 0.8 μg/kg; or about 2.3 μg/kg and about 2.5 μg/kg; or about 6.5 μg/kg and about 8.5 μg/kg; or about 18 μg/kg and about 22 μg/kg; or about 40 μg/kg and about 50 μg/kg; or about 75 μg/kg and about 85 μg/kg; or about 120 μg/kg and about 130 μg/kg; or between about 165 μg/kg and about 175 μg/kg.

In an exemplary embodiment, the bispecific anti-CD20×anti-CD3 antibody (e.g., XENP13676) is administered intravenously by infusion every other week in an amount of from about 0.7 μg/kg to about 170 μg/kg, e.g., about 2.4 μg/kg to about 170 μg/kg, about 7.5 μg/kg to about 170 μg/kg, about 20 μg/kg to about 170 μg/kg, about 45 μg/kg to about 170 μg/kg, about 80 μg/kg to about 170 μg/kg, about 125 μg/kg to about 170 μg/kg, about 0.7 μg/kg to about 125 μg/kg, about 2.4 μg/kg to about 125 μg/kg, about 7.5 μg/kg to about 125 μg/kg, about 20 μg/kg to about 125 μg/kg, about 45 μg/kg to about 125 μg/kg, about 80 μg/kg to about 125 μg/kg, about 0.7 μg/kg to about 80 μg/kg, about 2.4 μg/kg to about 80 μg/kg, about 7.5 μg/kg to about 80 μg/kg, about 20 μg/kg to about 80 μg/kg, about 45 μg/kg to about 80 μg/kg, about 0.7 μg/kg to about 45 μg/kg, about 2.4 μg/kg to about 45 μg/kg, about 7.5 μg/kg to about 45 μg/kg, about 20 μg/kg to about 45 μg/kg, about 0.7 μg/kg to about 20 μg/kg, about 2.4 μg/kg to about 20 μg/kg, about 7.5 μg/kg to about 20 μg/kg, about 0.7 μg/kg to about 7.5 μg/kg, about 2.4 μg/kg to about 7.5 μg/kg, about 0.7 μg/kg to about 2.4 μg/kg. In an exemplary embodiment, the bispecific anti-CD20×anti-CD3 antibody (e.g., XENP13676) is administered intravenously by infusion every other week in an amount of from about 0.6 μg/kg and about 0.8 μg/kg; or about 2.3 μg/kg and about 2.5 μg/kg; or about 6.5 μg/kg and about 8.5 μg/kg; or about 18 μg/kg and about 22 μg/kg; or about 40 μg/kg and about 50 μg/kg; or about 75 μg/kg and about 85 μg/kg; or about 120 μg/kg and about 130 μg/kg; or between about 165 μg/kg and about 175 μg/kg.

In an exemplary embodiment, the bispecific anti-CD20×anti-CD3 antibody (e.g., XENP13676) is administered intravenously by infusion weekly in an amount of from about 0.7 μg/kg to about 170 μg/kg, e.g., about 2.4 μg/kg to about 170 μg/kg, about 7.5 μg/kg to about 170 μg/kg, about 20 μg/kg to about 170 μg/kg, about 45 μg/kg to about 170 μg/kg, about 80 μg/kg to about 170 μg/kg, about 125 μg/kg to about 170 μg/kg, about 0.7 μg/kg to about 125 μg/kg, about 2.4 μg/kg to about 125 μg/kg, about 7.5 μg/kg to about 125 μg/kg, about 20 μg/kg to about 125 μg/kg, about 45 μg/kg to about 125 μg/kg, about 80 μg/kg to about 125 μg/kg, about 0.7 μg/kg to about 80 μg/kg, about 2.4 μg/kg to about 80 μg/kg, about 7.5 μg/kg to about 80 μg/kg, about 20 μg/kg to about 80 μg/kg, about 45 μg/kg to about 80 μg/kg, about 0.7 μg/kg to about 45 μg/kg, about 2.4 μg/kg to about 45 μg/kg, about 7.5 μg/kg to about 45 μg/kg, about 20 μg/kg to about 45 μg/kg, about 0.7 μg/kg to about 20 μg/kg, about 2.4 μg/kg to about 20 μg/kg, about 7.5 μg/kg to about 20 μg/kg, about 0.7 μg/kg to about 7.5 μg/kg, about 2.4 μg/kg to about 7.5 μg/kg, about 0.7 μg/kg to about 2.4 μg/kg. In an exemplary embodiment, the bispecific anti-CD20×anti-CD3 antibody (e.g., XENP13676) is administered intravenously by infusion weekly in an amount of from about 0.6 μg/kg and about 0.8 μg/kg; or about 2.3 μg/kg and about 2.5 μg/kg; or about 6.5 μg/kg and about 8.5 μg/kg; or about 18 μg/kg and about 22 μg/kg; or about 40 μg/kg and about 50 μg/kg; or about 75 μg/kg and about 85 μg/kg; or about 120 μg/kg and about 130 μg/kg; or between about 165 μg/kg and about 175 μg/kg.

In an exemplary embodiment, the bispecific anti-CD20×anti-CD3 antibody (e.g., XENP13676) is administered intravenously by infusion monthly in an amount of from about 0.45 μg/kg to about 110 μg/kg, e.g., about 1.6 μg/kg to about 110 μg/kg, about 5 μg/kg to about 110 μg/kg, about 12.5 μg/kg to about 110 μg/kg, about 28 μg/kg to about 110 μg/kg, about 5 μg/kg to about 80 μg/kg, about 12.5 μg/kg to about 80 μg/kg, about 28 μg/kg to about 80 μg/kg, about 50 μg/kg to about 80 μg/kg, about 28 μg/kg to about 50 μg/kg, about 28 μg/kg to about 100 μg/kg, about 28 μg/kg to about 90 μg/kg, about 28 μg/kg to about 70 μg/kg, about 28 μg/kg to about 60 μg/kg, about 28 μg/kg to about 50 μg/kg, about 28 μg/kg to about 40 μg/kg, about 30 μg/kg to about 80 μg/kg, about 40 μg/kg to about 80 μg/kg, about 50 μg/kg to about 80 μg/kg, about 60 μg/kg to about 80 μg/kg, about 70 μg/kg to about 80 μg/kg, about 40 μg/kg to about 70 μg/kg, about 40 μg/kg to about 60 μg/kg, about 40 μg/kg to about 50 μg/kg, about 50 μg/kg to about 70 μg/kg, about 50 μg/kg to about 60 μg/kg.

In an exemplary embodiment, the bispecific anti-CD20×anti-CD3 antibody (e.g., XENP13676) is administered intravenously by infusion every other week in an amount of from about 0.45 μg/kg to about 110 μg/kg, e.g., about 1.6 μg/kg to about 110 μg/kg, about 5 μg/kg to about 110 μg/kg, about 12.5 μg/kg to about 110 μg/kg, about 28 μg/kg to about 110 μg/kg, about 5 μg/kg to about 80 μg/kg, about 12.5 μg/kg to about 80 μg/kg, about 28 μg/kg to about 80 μg/kg, about 50 μg/kg to about 80 μg/kg, about 28 μg/kg to about 50 μg/kg, about 28 μg/kg to about 100 μg/kg, about 28 μg/kg to about 90 μg/kg, about 28 μg/kg to about 70 μg/kg, about 28 μg/kg to about 60 μg/kg, about 28 μg/kg to about 50 μg/kg, about 28 μg/kg to about 40 μg/kg, about 30 μg/kg to about 80 μg/kg, about 40 μg/kg to about 80 μg/kg, about 50 μg/kg to about 80 μg/kg, about 60 μg/kg to about 80 μg/kg, about 70 μg/kg to about 80 μg/kg, about 40 μg/kg to about 70 μg/kg, about 40 μg/kg to about 60 μg/kg, about 40 μg/kg to about 50 μg/kg, about 50 μg/kg to about 70 μg/kg, about 50 μg/kg to about 60 μg/kg.

In an exemplary embodiment, the bispecific anti-CD20×anti-CD3 antibody (e.g., XENP13676) is administered intravenously by infusion weekly in an amount of from about 0.45 μg/kg to about 110 μg/kg, e.g., about 1.6 μg/kg to about 110 μg/kg, about 5 μg/kg to about 110 μg/kg, about 12.5 μg/kg to about 110 μg/kg, about 28 μg/kg to about 110 μg/kg, about 5 μg/kg to about 80 μg/kg, about 12.5 μg/kg to about 80 μg/kg, about 28 μg/kg to about 80 μg/kg, about 50 μg/kg to about 80 μg/kg, about 28 μg/kg to about 50 μg/kg, about 28 μg/kg to about 100 μg/kg, about 28 μg/kg to about 90 μg/kg, about 28 μg/kg to about 70 μg/kg, about 28 μg/kg to about 60 μg/kg, about 28 μg/kg to about 50 μg/kg, about 28 μg/kg to about 40 μg/kg, about 30 μg/kg to about 80 μg/kg, about 40 μg/kg to about 80 μg/kg, about 50 μg/kg to about 80 μg/kg, about 60 μg/kg to about 80 μg/kg, about 70 μg/kg to about 80 μg/kg, about 40 μg/kg to about 70 μg/kg, about 40 μg/kg to about 60 μg/kg, about 40 μg/kg to about 50 μg/kg, about 50 μg/kg to about 70 μg/kg, about 50 μg/kg to about 60 μg/kg.

In an exemplary embodiment, the bispecific anti-CD20×anti-CD3 antibody (e.g., XENP13676) is administered by infusion for a period of between about one hour and about three hours. In an exemplary embodiment, the bispecific anti-CD20×anti-CD3 antibody (e.g., XENP13676) is administered by infusion for a period of about two hours. In an exemplary embodiment, the bispecific anti-CD20×anti-CD3 antibody (e.g., XENP13676) is administered by infusion for a period of two hours.

In an exemplary embodiment, the bispecific anti-CD20×anti-CD3 antibody (e.g., XENP13676) is administered once every 6-8 days for between about 1 and about 9 weeks. In an exemplary embodiment, the bispecific anti-CD20×anti-CD3 antibody (e.g., XENP13676) is administered once every 6-8 days for between about 2 and about 7 weeks. In an exemplary embodiment, the bispecific anti-CD20×anti-CD3 antibody (e.g., XENP13676) is administered once every 6-8 days for between about 3 and about 9 weeks. In an exemplary embodiment, the bispecific anti-CD20×anti-CD3 antibody (e.g., XENP13676) is administered once every 6-8 days for between about 1 and about 8 weeks. In an exemplary embodiment, the bispecific anti-CD20×anti-CD3 antibody (e.g., XENP13676) is administered once every 6-8 days for between about 3 and about 5 weeks. In an exemplary embodiment, the bispecific anti-CD20×anti-CD3 antibody (e.g., XENP13676) is administered once every 6-8 days for about 4 weeks. In an exemplary embodiment, the bispecific anti-CD20×anti-CD3 antibody (e.g., XENP13676) is administered once every 6-8 days for 4 weeks. In an exemplary embodiment, the bispecific anti- anti-CD20×anti-CD3 antibody (e.g., XENP13676) is administered once every 6-8 days for between about 7 and about 9 weeks. In an exemplary embodiment, the bispecific anti-CD20×anti-CD3 antibody (e.g., XENP13676) is administered once every 6-8 days for about 8 weeks. In an exemplary embodiment, the bispecific anti-CD20×anti-CD3 antibody (e.g., XENP13676) is administered once every 6-8 days for 8 weeks.

The dosage may be determined or adjusted by measuring the amount of bispecific anti-CD20×anti-CD3 antibody of the present invention (e.g., XENP13676) in the blood upon administration using techniques known in the art, for instance taking out a biological sample and using anti-idiotypic antibodies which target the antigen binding region of the bispecific anti-CD20×anti-CD3 antibody (e.g., XENP13676).

In an exemplary embodiment, the amount is between about 0.1 μg/kg and about 200 μg/kg.

In an exemplary embodiment, the amount is between about 0.1 μg/kg and about 1 μg/kg. In an exemplary embodiment, the amount is between about 0.25 μg/kg and about 0.75 μg/kg. In an exemplary embodiment, the amount is between about 0.35 μg/kg and about 0.55 μg/kg. In an exemplary embodiment, the amount is about 0.45 μg/kg. In an exemplary embodiment, the amount is 0.45 μg/kg.

In an exemplary embodiment, the amount is between about 0.2 μg/kg and about 1.2 μg/kg. In an exemplary embodiment, the amount is between about 0.3 μg/kg and about 1.1 μg/kg. In an exemplary embodiment, the amount is between about 0.4 μg/kg and about 1.0 μg/kg. In an exemplary embodiment, the amount is between about 0.5 μg/kg and about 0.9 μg/kg. In an exemplary embodiment, the amount is between about 0.6 μg/kg and about 0.8 μg/kg. In an exemplary embodiment, the amount is between about 0.65 μg/kg and about 0.75 μg/kg. In an exemplary embodiment, the amount is about 0.7 μg/kg. In an exemplary embodiment, the amount is 0.7 μg/kg.

In an exemplary embodiment, the amount is between about 1 μg/kg and about 2 μg/kg. In an exemplary embodiment, the amount is between about 1.25 μg/kg and about 1.75 μg/kg. In an exemplary embodiment, the amount is between about 1.4 μg/kg and about 1.7 μg/kg. In an exemplary embodiment, the amount is about 1.6 μg/kg. In an exemplary embodiment, the amount is 1.60 μg/kg.

In an exemplary embodiment, the amount is between about 1.9 μg/kg and about 2.9 μg/kg. In an exemplary embodiment, the amount is between about 2.0 μg/kg and about 2.8 μg/kg. In an exemplary embodiment, the amount is between about 2.1 μg/kg and about 2.7 μg/kg. In an exemplary embodiment, the amount is between about 2.2 μg/kg and about 2.6 μg/kg. In an exemplary embodiment, the amount is between about 2.3 μg/kg and about 2.5 μg/kg. In an exemplary embodiment, the amount is between about 2.35 μg/kg and about 2.45 μg/kg. In an exemplary embodiment, the amount is about 2.4 μg/kg. In an exemplary embodiment, the amount is 2.4 μg/kg.

In an exemplary embodiment, the amount is between about 1 μg/kg and about 10 μg/kg. In an exemplary embodiment, the amount is between about 2 μg/kg and about 8 μg/kg. In an exemplary embodiment, the amount is between about 3 μg/kg and about 7 μg/kg. In an exemplary embodiment, the amount is between about 4 μg/kg and about 6 μg/kg. In an exemplary embodiment, the amount is about 5 μg/kg. In an exemplary embodiment, the amount is 5 μg/kg.

In an exemplary embodiment, the amount is between about 2.5 μg/kg and about 12.5 μg/kg. In an exemplary embodiment, the amount is between about 3.5 μg/kg and about 11.5 μg/kg. In an exemplary embodiment, the amount is between about 4.5 μg/kg and about 10.5 μg/kg. In an exemplary embodiment, the amount is between about 5.5 μg/kg and about 9.5 μg/kg. In an exemplary embodiment, the amount is between about 6.5 μg/kg and about 8.5 μg/kg. In an exemplary embodiment, the amount is between about 7.0 μg/kg and about 8.0 μg/kg. In an exemplary embodiment, the amount is about 7.5 μg/kg. In an exemplary embodiment, the amount is 7.5 μg/kg.

In an exemplary embodiment, the amount is between about 7.5 μg/kg and about 17.50 μg/kg. In an exemplary embodiment, the amount is between about 10 μg/kg and about 15 μg/kg. In an exemplary embodiment, the amount is between about 11 μg/kg and about 14 μg/kg. In an exemplary embodiment, the amount is between about 12 μg/kg and about 13 μg/kg. In an exemplary embodiment, the amount is about 12.5 μg/kg. In an exemplary embodiment, the amount is 12.5 μg/kg.

In an exemplary embodiment, the amount is between about 10 μg/kg and about 30 μg/kg. In an exemplary embodiment, the amount is between about 12 μg/kg and about 28 μg/kg. In an exemplary embodiment, the amount is between about 14 μg/kg and about 26 μg/kg. In an exemplary embodiment, the amount is between about 16 μg/kg and about 24 μg/kg. In an exemplary embodiment, the amount is between about 18 μg/kg and about 22 μg/kg. In an exemplary embodiment, the amount is between about 19 μg/kg and about 21 μg/kg. In an exemplary embodiment, the amount is about 20 μg/kg. In an exemplary embodiment, the amount is 20 μg/kg.

In an exemplary embodiment, the amount is between about 10 μg/kg and about 50 μg/kg. In an exemplary embodiment, the amount is between about 15 μg/kg and about 45 μg/kg. In an exemplary embodiment, the amount is between about 20 μg/kg and about 40 μg/kg. In an exemplary embodiment, the amount is between about 25 μg/kg and about 32 μg/kg. In an exemplary embodiment, the amount is about 28 μg/kg. In an exemplary embodiment, the amount is 28 μg/kg.

In an exemplary embodiment, the amount is between about 15 μg/kg and about 65 μg/kg. In an exemplary embodiment, the amount is between about 20 μg/kg and about 60 μg/kg. In an exemplary embodiment, the amount is between about 25 μg/kg and about 55 μg/kg. In an exemplary embodiment, the amount is between about 30 μg/kg and about 50 μg/kg. In an exemplary embodiment, the amount is between about 35 μg/kg and about 50 μg/kg. In an exemplary embodiment, the amount is between about 40 μg/kg and about 50 μg/kg. In an exemplary embodiment, the amount is between about 42 μg/kg and about 48 μg/kg. In an exemplary embodiment, the amount is about 45 μg/kg. In an exemplary embodiment, the amount is 45 μg/kg.

In an exemplary embodiment, the amount is between about 25 μg/kg and about 75 μg/kg. In an exemplary embodiment, the amount is between about 35 μg/kg and about 65 μg/kg. In an exemplary embodiment, the amount is between about 40 μg/kg and about 60 μg/kg. In an exemplary embodiment, the amount is between about 45 μg/kg and about 55 μg/kg. In an exemplary embodiment, the amount is about 50 μg/kg. In an exemplary embodiment, the amount is 50 μg/kg.

In an exemplary embodiment, the amount is between about 20 μg/kg and about 140 μg/kg. In an exemplary embodiment, the amount is between about 40 μg/kg and about 120 μg/kg. In an exemplary embodiment, the amount is between about 45 μg/kg and about 115 μg/kg. In an exemplary embodiment, the amount is between about 50 μg/kg and about 110 μg/kg. In an exemplary embodiment, the amount is between about 55 μg/kg and about 105 μg/kg. In an exemplary embodiment, the amount is between about 60 μg/kg and about 100 μg/kg. In an exemplary embodiment, the amount is between about 65 μg/kg and about 95 μg/kg. In an exemplary embodiment, the amount is between about 70 μg/kg and about 90 μg/kg. In an exemplary embodiment, the amount is between about 75 μg/kg and about 85 μg/kg. In an exemplary embodiment, the amount is about 80 μg/kg. In an exemplary embodiment, the amount is 80 μg/kg. In an exemplary embodiment, the amount is between about 75 μg/kg and about 85 μg/kg. In an exemplary embodiment, the amount is about 80 μg/kg. In an exemplary embodiment, the amount is 80 μg/kg.

In an exemplary embodiment, the amount is between about 65 μg/kg and about 175 μg/kg. In an exemplary embodiment, the amount is between about 75 μg/kg and about 165 μg/kg. In an exemplary embodiment, the amount is between about 85 μg/kg and about 155 μg/kg. In an exemplary embodiment, the amount is between about 95 μg/kg and about 145 μg/kg. In an exemplary embodiment, the amount is between about 105 μg/kg and about 135 μg/kg. In an exemplary embodiment, the amount is between about 115 μg/kg and about 135 μg/kg. In an exemplary embodiment, the amount is between about 120 μg/kg and about 130 μg/kg. In an exemplary embodiment, the amount is about 125 μg/kg. In an exemplary embodiment, the amount is 125 μg/kg.

In an exemplary embodiment, the amount is between about 140 μg/kg and about 200 μg/kg. In an exemplary embodiment, the amount is between about 145 μg/kg and about 195 μg/kg. In an exemplary embodiment, the amount is between about 150 μg/kg and about 190 μg/kg. In an exemplary embodiment, the amount is between about 155 μg/kg and about 185 μg/kg. In an exemplary embodiment, the amount is between about 160 μg/kg and about 180 μg/kg. In an exemplary embodiment, the amount is between about 165 μg/kg and about 175 μg/kg. In an exemplary embodiment, the amount is about 170 μg/kg. In an exemplary embodiment, the amount is 170 μg/kg.

In an exemplary embodiment, prior to the administration of the bispecific anti-CD20×anti-CD3 antibody (e.g., XENP13676), the human subject is administered a steroid. In an exemplary embodiment, the human subject is administered the steroid between about 30 minutes and about 90 minutes prior to the administration of the bispecific anti-CD20×anti-CD3 antibody (e.g., XENP13676). In an exemplary embodiment, the human subject is administered the steroid about 60 minutes prior to the administration of the bispecific anti-CD20×anti-CD3 antibody (e.g., XENP13676). In an exemplary embodiment, the steroid is dexamethasone. In an exemplary embodiment, between about 10 mg and about 30 mg of dexamethasone is administered to the human subject. In an exemplary embodiment, about 20 mg of dexamethasone is administered to the human subject.

In an exemplary embodiment, the bispecific anti-CD20×anti-CD3 antibody (e.g., XENP13676) is administered intravenously. In an exemplary embodiment, the bispecific anti-CD20×anti-CD3 antibody (e.g., XENP13676) is administered weekly until disease progression, unacceptable toxicity, or individual choice.

In some embodiments, the bispecific anti-CD20×anti-CD3 antibody (e.g., XENP13676) is a front line therapy, second line therapy, third line therapy, fourth line therapy, fifth line therapy, or sixth line therapy.

In some embodiments, the bispecific anti-CD20×anti-CD3 antibody (e.g., XENP13676) treats a refractory lymphoma. In some embodiments, the bispecific anti-CD20×anti-CD3 antibody (e.g., XENP13676) is a maintenance therapy.

A medical professional having ordinary skill in the art may readily determine and prescribe the effective amount of the antibody composition required. For example, a physician could start doses of the medicament employed in the antibody composition at levels lower than that required in order to achieve the desired therapeutic effect and gradually increase the dosage until the desired effect is achieved.

IX. Treatment modalities

In the methods of the invention, treatment is used to provide a positive therapeutic response with respect to a lymphoma. By “positive therapeutic response” is intended an improvement in the lymphoma, and/or an improvement in the symptoms associated with the lymphoma. For example, a positive therapeutic response would refer to one or more of the following improvements in the lymphoma: (1) a reduction in the number of CD20⁺ lymphoma-associated cells; (2) an increase in CD20⁺ lymphoma-associated cell death; (3) inhibition of CD20⁺ lymphoma-associated cell survival; (5) inhibition (i.e., slowing to some extent, preferably halting) of CD20⁺ cell proliferation; (6) an increased patient survival rate; and (7) some relief from one or more symptoms associated with the lymphoma.

Positive therapeutic responses in any given lymphoma can be determined by standardized response criteria specific to that disease or condition.

In addition to these positive therapeutic responses, the subject undergoing treatment may experience the beneficial effect of an improvement in the symptoms associated with the lymphoma. In an exemplary embodiment, a treatment of lymphoma is selected from the group consisting of feeling less tired, feeling less weak, feeling less dizzy or lightheaded, reduction in shortness of breath, reduction in fever, quicker response to infections, reduction in ease of bruising, reduction in bleeding episodes, weight gain, reduction in night sweats, gain of appetite, reduction in abdominal swelling, reduction in lymph node swelling, reduction in bone or joint pain, and reduction in thymus swelling.

An improvement in the lymphoma may be characterized as a complete response. By “complete response” is intended an absence of clinically detectable disease with normalization of any previously abnormal radiographic studies, bone marrow, and cerebrospinal fluid (CSF) or abnormal monoclonal protein in the case of myeloma.

Such a response may persist for at least 4 to 8 weeks, or sometimes 6 to 8 weeks, following treatment according to the methods of the invention. Alternatively, an improvement in the lymphoma may be categorized as being a partial response. By “partial response” is intended at least about a 50% decrease in all measurable tumor burden (i.e., the number of malignant cells present in the subject, or the measured bulk of tumor masses or the quantity of abnormal monoclonal protein) in the absence of new lesions, which may persist for 4 to 8 weeks, or 6 to 8 weeks.

Treatment according to the present invention includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody are outweighed by the therapeutically beneficial effects.

A “therapeutically effective amount” for therapy may also be measured by its ability to stabilize the progression of the lymphoma. The ability of an antibody to inhibit lymphoma may be evaluated in an animal model system predictive of efficacy in a human.

Alternatively, this property of an antibody composition may be evaluated by examining the ability of the antibody to inhibit cell growth or to induce apoptosis by in vitro assays known to the skilled practitioner. A therapeutically effective amount of a bispecific anti-CD20×anti-CD3 antibody (e.g., XENP13676) may reduce the number of CD20⁺ lymphoma-associated cells, or improve other aspects related to the lymphoma (such as those described herein), and/or otherwise ameliorate symptoms in a human subject (such as those also described herein). One of ordinary skill in the art would be able to determine such amounts based on such factors as the subject's size, the severity of the subject's symptoms, and the particular antibody composition or route of administration selected.

### X. Combination Therapy

In certain instances, a bispecific anti-CD20×anti-CD3 antibody (e.g., XENP13676) described herein can be used in combination with another therapeutic agent. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The bispecific anti-CD20×anti-CD3 antibody (e.g., XENP13676) described herein and the at least one additional therapeutic agent can be administered simultaneously, in the same or in separate compositions, or sequentially. For sequential administration, the bispecific anti-CD20×anti-CD3 antibody (e.g., XENP13676) described herein can be administered first, and the additional agent can be administered second, or the order of administration can be reversed.

The bispecific anti-CD20×anti-CD3 antibody (e.g., XENP13676) and/or other therapeutic agents, procedures or modalities can be administered during periods of active disorder, or during a period of remission or less active disease. The bispecific anti-CD20×anti-CD3 antibody (e.g., XENP13676) can be administered before the other treatment, concurrently with the treatment, post-treatment, or during remission of the disorder.

When administered in combination, the bispecific anti-CD20×anti-CD3 antibody (e.g., XENP13676) and the additional agent (e.g., second or third agent), or all, can be administered in an amount or dose that is lower or the same than the amount or dosage of each agent used individually, e.g., as a monotherapy. In some embodiments, the administered amount or dosage of the bispecific anti-CD20×anti-CD3 antibody (e.g., XENP13676), the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the bispecific anti-CD20×anti-CD3 antibody (e.g., XENP13676), the additional agent (e.g., second or third agent), or all, that results in a desired effect (e.g., treatment of cancer) is lower (e.g., at least 20%, at least 30%, at least 40%, or at least 50% lower) than the amount or dosage of each agent used individually, e.g., as a monotherapy, required to achieve the same therapeutic effect.

In further aspects, a bispecific anti-CD20×anti-CD3 antibody (e.g., XENP13676) described herein may be used in a treatment regimen in combination with chemotherapy, radiation, immunosuppressive agents, such as cyclosporin, azathioprine, methotrexate, mycophenolate, and FK506, or other immunoablative agents such as CAMPATH, other antibody therapies, cytoxan, fludarabine, cyclosporin, FK506, rapamycin, mycophenolic acid, steroids, FR90165, cytokines, and irradiation. peptide vaccine, such as that described in Izumoto et al. 2008 J Neurosurg 108:963-971.

In certain instances, compounds of the present invention are combined with other therapeutic agents, such as other anti-cancer agents, anti-allergic agents, anti-nausea agents (or anti-emetics), pain relievers, cytoprotective agents, and combinations thereof.

In one embodiment, a bispecific anti-CD20×anti-CD3 antibody (e.g., XENP13676) described herein can be used in combination with a chemotherapeutic agent. Exemplary chemotherapeutic agents include an anthracycline (e.g., idarubicin, daunorubicin, doxorubicin (e.g., liposomal doxorubicin)), a anthracenedione derivative (e.g., mitoxantrone), a vinca alkaloid (e.g., vinblastine, vincristine, vindesine, vinorelbine), an alkylating agent (e.g., cyclophosphamide, dacarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, cytarabine, pyrimidine analogs, purine analogs and adenosine deaminase inhibitors (e.g., fludarabine)), an mTOR inhibitor, a proteasome inhibitor (e.g., aclacinomycin A, gliotoxin or bortezomib), an immunomodulator such as thalidomide or a thalidomide derivative (e.g., lenalidomide), a kinase inhibitor such as ibrutinib (e.g., Imbruvica), a corticosteroid (e.g., dexamethasone, prednisone), and CVP (a combination of cyclophosphamide, vincristine, and prednisone), CHOP (a combination of cyclophosphamide, hydroxydaunorubicin, Oncovin® (vincristine), and prednisone) with or without etoposide (e.g., VP-16), a combination of cyclophosphamide and pentostatin, a combination of chlorambucil and prednisone, a combination of fludarabine and cyclophosphamide, or another agent such as mechlorethamine hydrochloride (e.g. Mustargen), doxorubicin (Adriamycin®), methotrexate, oxaliplatin, or cytarabine (ara-C).

General Chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

In some embodiments, a bispecific anti-CD20×anti-CD3 antibody (e.g., XENP13676) described herein is administered to a subject in combination with one or more of the following agents: an anti-TNF antibody, a steroid, or an antihistamine (e.g., Benadryl).

In some embodiments, a bispecific anti-CD20×anti-CD3 antibody (e.g., XENP13676) described herein is administered to a subject who has CLL/SLL, in combination with one or more of the following agents: an alkylating agent such as bendamustine hydrochloride (e.g., Treanda), chlorambucil (e.g., Leukeran, Ambochlorin, Amboclorin, Linfolizin), cyclophosphamide (e.g., Cytoxan, Clafen, Neosar); a purine analog such as fludarabine phosphate (e.g., Fludara), cladribine (e.g., Leustatin, 2-CdA), pentostatin (Nipent®); an Bcl2 inhibitor such as ABT-737, venetoclax (e.g., Venclexta); a kinase inhibitor such as ibrutinib (e.g., Imbruvica), venetoclax, idelalisib (e.g., Zydelig); an anti-CD52 Ab such as alemtuzumab (Campath®); a corticosteroid such as prednisone, methylprednisolone, or dexamethasone; or CVP (a combination of cyclophosphamide, vincristine, and prednisone), CHOP (a combination of cyclophosphamide, hydroxydaunorubicin, Oncovin® (vincristine), and prednisone) with or without etoposide (e.g., VP-16), a combination of cyclophosphamide and pentostatin, a combination of chlorambucil and prednisone, a combination of fludarabine and cyclophosphamide, or another agent such as mechlorethamine hydrochloride (e.g. Mustargen), doxorubicin (Adriamycin®), methotrexate, oxaliplatin, or cytarabine (ara-C).

In one embodiment, the subject can be administered an agent which reduces or ameliorates a side effect associated with the administration of a bispecific anti-CD20×anti-CD3 antibody (e.g., XENP13676). Side effects associated with the administration of a bispecific anti-CD20×anti-CD3 antibody (e.g., XENP13676) include, but are not limited to, cytokine release syndrome (“CRS”) and hemophagocytic lymphohistiocytosis (HLH), also termed Macrophage Activation Syndrome (MAS). Symptoms of CRS may include high fevers, nausea, transient hypotension, hypoxia, and the like. CRS may include clinical constitutional signs and symptoms such as fever, fatigue, anorexia, myalgias, arthalgias, nausea, vomiting, and headache. CRS may include clinical skin signs and symptoms such as rash. CRS may include clinical gastrointestinal signs and symsptoms such as nausea, vomiting and diarrhea. CRS may include clinical respiratory signs and symptoms such as tachypnea and hypoxemia. CRS may include clinical cardiovascular signs and symptoms such as tachycardia, widened pulse pressure, hypotension, increased cardac output (early) and potentially diminished cardiac output (late). CRS may include clinical coagulation signs and symptoms such as elevated d-dimer, hypofibrinogenemia with or without bleeding. CRS may include clinical renal signs and symptoms such as azotemia. CRS may include clinical hepatic signs and symptoms such as transaminitis and hyperbilirubinemia. CRS may include clinical neurologic signs and symptoms such as headache, mental status changes, confusion, delirium, word finding difficulty or frank aphasia, hallucinations, tremor, dymetria, altered gait, and seizures. In one embodiment, the subject is administered an agent that reduces an immune-mediated side effect. Exemplary immune-mediated side effects include, but are not limited to pneumonitis, colitis, hepatitis, nephristis and renal disfunction, hypothyroidism, hyperthyroidism, and endocrinopathies (e.g., hypophysitis, Type 1 diabetes mellitus and thyroid disorders such as hypothyroidism and hyperthyroidism). In one embodiment, the subject is administered an agent that reduces embryofetal toxicity. In some embodiments, the subject is administered an agent that reduces embryofetal toxicity

Accordingly, the methods described herein can comprise administering a bispecific anti-CD20×anti-CD3 antibody (e.g., XENP13676) described herein to a subject and further administering one or more agents to manage elevated levels of a soluble factor resulting from treatment with a bispecific anti-CD20×anti-CD3 antibody (e.g., XENP13676). In one embodiment, the soluble factor elevated in the subject is one or more of IFN-γ, TNFα, IL-2 and IL-6. In an embodiment, the factor elevated in the subject is one or more of IL-1, GM-CSF, IL-10, IL-8, IL-5 and fraktalkine. Therefore, an agent administered to treat this side effect can be an agent that neutralizes one or more of these soluble factors. In one embodiment, the agent that neutralizes one or more of these soluble forms is an antibody or antigen binding fragment thereof. Examples of such agents include, but are not limited to a steroid (e.g., corticosteroid), an inhibitor of TNFα, and inhibitor of IL-1R, and an inhibitor of IL-6. An example of a TNFα inhibitor is an anti-TNFα antibody molecule such as, infliximab, adalimumab, certolizumab pegol, and golimumab. Another example of a TNFα inhibitor is a fusion protein such as entanercept. Small molecule inhibitor of TNFα include, but are not limited to, xanthine derivatives (e.g. pentoxifylline) and bupropion. An example of an IL-6 inhibitor is an anti-IL-6 antibody molecule such as tocilizumab (toc), sarilumab, elsilimomab, CNTO 328, ALD518/BMS-945429, CNTO 136, CPSI-2364, CDP6038, VX30, ARGX-109, FE301, and FM101. In one embodiment, the anti-IL-6 antibody molecule is tocilizumab. An example of an IL-1R based inhibitor is anakinra.

In some embodiment, the subject is administered a corticosteroid, such as, e.g., methylprednisolone, hydrocortisone, among others. In some embodiments, the subject is administered a corticosterioid, e.g., methylprednisolone, hydrocortisone, in combination with Benadryl and Tylenol prior to the administration of a anti-CD20×anti-CD3 antibody (e.g., XENP13676) to mitigate the CRS risk.

In some embodiments, the subject is administered a vasopressor, such as, e.g., norepinephrine, dopamine, phenylephrine, epinephrine, vasopressin, or a combination thereof

In an embodiment, the subject can be administered an antipyretic agent. In an embodiment, the subject can be administered an analgesic agent.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

### EXAMPLES

Examples are provided below to illustrate the present invention. These examples are not meant to constrain the present invention to any particular application or theory of operation. For all constant region positions discussed in the present invention, numbering is according to the EU index as in Kabat (Kabat et al., 1991, Sequences of Proteins of Immunological Interest, 5th Ed., United States Public Health Service, National Institutes of Health, Bethesda, entirely incorporated by reference). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

General and specific scientific techniques are outlined in U.S. Publications 2015/0307629, and 2014/0288275, as well as PCT Publication WO2014/145806, as well as U.S. Applications 62/085,027, Ser. No. 14/952,714, and Ser. No. 15/141,350, all of which are expressly incorporated by reference in their entirety and particularly for the techniques outlined therein.

### Example 1

### XENP13676 Treatment Plan

This is a multi-dose Phase 1 dose-escalation study designed to define a maximal tolerated dose and schedule, to preliminarily describe safety, and to assess PK, immunogenicity, and potential anti-tumor activity of XENP13676 in patients with relapsed or refractory B-cell malignancies.

This study will enroll two parallel groups of patients: dosing cohorts that establish an MTD/RD in patients with non-CLL B-cell malignancies (Group NHL) and dosing cohorts that establish an MTD/RD for patients with CLL/SLL (Group CLL).

In both groups, escalation to higher dose cohorts will be made based on Dose Escalation Review Committee (DERC) review of the aggregate safety data through at least Day 22 on all patients participating in previous cohorts. The DERC will be allowed to make adaptations to the dosing schema if felt to be needed, in accordance with evolving trial data regarding the dosing regimen and in the spirit of the current study protocol (i.e., not significantly affecting the current risk profile of the study).

Once the MTD/RDs are established, the groups will be expanded with the addition of up to 6-12 patients to increase the safety experience and more extensively evaluate the PK and PD of XENP13676.

Patients will be assessed for tumor response every 8 weeks while on study drug. Patients who appear to benefit from XENP13676 treatment and continue to meet eligibility may continue treatment past the initial 8 weekly doses (two cycles). If patients tolerate their initial dose level and there have been no DLTs on the next higher dose level after all patients have completed the DLT period, they may be treated at the higher dose of XENP13676.

### Number of Subjects

Up to 65 subjects will be enrolled; the total number required will depend on the number of dose cohorts required to define the MTD in both groups. Up to 10 clinical investigation sites will enroll patients to this study.

### Dose Escalation Scheme

Patients will be enrolled in parallel to both the NHL and CLL Groups, and dose escalation in each group will be essentially independent. Dose level increases will initially proceed according to an accelerated titration design (see Table 1 and Table 2). This design allows for more efficient dose escalation while maintaining safety standards by implementing conservative triggers for cohort expansion during the accelerated escalation phase, and may limit the number of patients exposed to potentially sub-therapeutic doses of XENP13676.

During the initial accelerated dose escalation phase (Cohorts 1N, 2N, 3N, 1C, 2C, and 3C), dose escalation may occur after treatment of 1 patient per cohort provided that there is no new ≧Grade 2 toxicity (i.e. no toxicity that existed prior to enrollment) during Cycle 1 and the patient has met minimum safety assessment requirements (see Table 2). When a patient experiences a ≧Grade 2 toxicity during the dose escalation safety assessment period, the accelerated escalation phase will end, the standard dose escalation phase will begin, and the cohort in which the event(s) occurred will be expanded to a total of at least 3 patients (2 additional patients will be enrolled).

TABLE 1
Dose Escalation Cohorts

| Group | Cohort | Planned Dose | Subjects |
|---|---|---|---|
| Non-CLL B cell | 1N | 0.45 μg/kg | 1 (+2 + 3) |
| malignancies | 2N | 1.6 μg/kg | 1 (+2 + 3) |
| (Group NHL) | 3N | 5.0 μg/kg | 1 (+2 + 3) |
| | 4N | 12.5 μg/kg | 3 (+3) |
| | 5N | 28 μg/kg | 3 (+3) |
| | 6N | 50 μg/kg | 3 (+3) |
| | 7N | 80 μg/kg | 3 (+3) |
| | 8N | 110 μg/kg | 3 (+3) |
| | Expansion-N | At MTD or recom- | 6-12 |
| | | mended dose | |
| CLL/SLL | 1C | 0.45 μg/kg | 1 (+2 + 3) |
| (Group CLL) | 2C | 1.6 μg/kg | 1 (+2 + 3) |
| | 3C | 5.0 μg/kg | 1 (+2 + 3) |
| | 4C | 12.5 μg/kg | 3 (+3) |
| | 5C | 28 μg/kg | 3 (+3) |
| | 6C | 50 μg/kg | 3 (+3) |
| | 7C | 80 μg/kg | 3 (+3) |
| | 8C | 110 μg/kg | 3 (+3) |
| | Expansion-C | At MTD or recom- | 6-12 |
| | | mended dose | |

MTD = maximum tolerated dose

TABLE 2
Dose Escalation Scheme
Accelerated Dose Escalation Phase

| | Number of Patients | |
|---|---|---|
| Number of Patients | Enrolled and Assessable for | |
| with at Least One | Safety Following Four | |
| Event ≧ Grade 2 | Doses of XENP13676 | Escalation Decision |
| 0 | 1 | Escalate to the next |
| | | higher dose level |
| 1 | 1 | Enroll 2 additional |
| | | patients on the same |
| | | dose level and revert |
| | | to Standard Dose |
| | | Escalation (3 + 3) |
| | | design below. |

Standard Dose Escalation Phase

| | Number of Patients | |
|---|---|---|
| Number of Patients | Enrolled and Assessable for | |
| with at Least One | Safety Following Four | |
| DLT | Doses of XENP13676 | Escalation Decision |
| 0 | 3 | Escalate to the next |
| | | higher dose level |
| 1 | 3 | Enroll 3 additional |
| | | patients on the same |
| | | dose level |
| 1 | 6 | Escalate to the next |
| | | higher dose level |
| 2 | 3 or 6 | No dose escalation |
| | | may occur; MTD has |
| | | been surpassed. The |
| | | next lower dose level |
| | | should be expanded. |

DLT = dose-limiting toxicity;
MTD = maximum tolerated dose

From this cohort forward (or beginning with Cohort 4N or 4C [21 μg/kg], whichever comes first) the standard 3+3 dose escalation rules will apply:

If zero of 3 patients have a DLT, then dose escalation to the next level will occur.

If 1 of 3 patients has a DLT, then the cohort will be further expanded to a total of 6 patients or until a second patient in the cohort experiences a DLT. If there are no additional patients with a DLT, then dose escalation to the next higher dose level will occur.

The MTD is defined as the highest dose level at which no more than 1 patient experiences DLT out of 6 patients assessable for toxicity at that dose level. Any cohort with 2 or more patients experiencing a DLT will have exceeded the MTD and there will be no further dose escalation. The dose level below the cohort at which 2 or more patients with DLT occurred will be expanded to at least 6 to delineate the MTD.

Before a dose-escalation decision can be reached, at least 1 patient (in the accelerated dose escalation phase of the study) or 3 patients (in the standard escalation phase of the study) must meet all requirements for dose escalation safety assessment.

For the purpose of determining the incidence of DLT and defining the MTD and/or recommended dosing of XENP13676 for future study, only patients who experience DLT and those with sufficient safety data/follow-up will be evaluated. Patients who complete 4 doses of XENP13676 and undergo the planned safety evaluations through Day 22 will be considered to have sufficient safety data/follow-up. Patients who withdraw from study before completing Day 22 of treatment for reasons unrelated to study drug toxicity will be considered to have inadequate data to support dose escalation. In such cases, replacement patients will be enrolled to receive the same dose of XENP13676 as the patients who withdraw prematurely.

The decision to advance dosing to the next cohort level will be made by the DERC after review of all required dose escalation safety assessment data from patients in a cohort. PK and ADA data may not be routinely available during the safety assessment period as these samples may be batched for analysis so that a more uniform drug exposure analysis and ADA analysis can be performed across all study samples. However, if a patient safety issue arises and the treating physician feels that information around drug exposure and/or ADA analysis would be useful information in determining the treatment plan for the patients, PK and ADA analysis may be performed on the patient samples that have been collected to date.

Once the MTD (or RD for further study) is identified, the MTD/RD dose level may be further expanded up to an additional 12 patients (up to a total MTD/RD cohort of 18 patients) to further assess safety and PK.

The dose escalation scheme may be modified (e.g., smaller increases or decreases in dose level may be permitted, additional patients in a cohort may be enrolled, infusion duration and scheduling may be modified) based on the type and severity of toxicities observed in this trial, upon agreement of the DERC. Enrolling additional patients beyond 65 requires a protocol amendment.

### Example 2

### XENP13676 Treatment Plan

This is a Phase 1, multiple-dose dose-escalation study designed to define a safe initial “priming dose” and a maximal tolerated dose and schedule, to describe safety and tolerability, to assess PK and immunogenicity, and to preliminarily assess potential anti-tumor activity of XENP13676 in patients with relapsed or refractory B cell malignancies.

This study will enroll two parallel Disease Groups of patients: dosing cohorts that establish a priming dose and maximal tolerated dose (MTD) or recommended dose (RD) and schedule in patients with non-CLL B cell malignancies (Group NHL) and dosing cohorts that establish a priming dose and MTD/RD and schedule for patients with CLL/SLL (Group CLL).

This study is designed in two parts:
Part A, escalating dose cohorts that establish an initial “priming dose” (the lowest initial dose with occurrence of a single DLT) as part of repeated weekly infusions at a fixed dose in a 28-day cycle; and
Part B, escalating dose cohorts that establish a MTD/RD for a dosing schedule consisting of a “priming dose” on Cycle 1 Day 1, established in Part A, followed by cohort escalation of fixed weekly infusions for Cycle 1 Day 7 through Cycle 2 Day 22.
   In both Disease Groups, escalation to higher dose cohorts will be made based on Dose Escalation Review Committee (DERC) review of the aggregate safety data through Cycle 1 Day 28 on all patients in Part A and through Cycle 2 Day 7 in Part B. In addition, the safety of the priming dose in Part B will be followed by continuous dose-limiting toxicity (DLT) assessment for 7 days following the priming dose in all Part B cohorts. The DERC will be allowed to make adaptations to the dosing schema if felt to be needed, in accordance with evolving trial safety and tolerability findings as long as changes do not significantly affect the risk profile of the study.

Once the MTD/RD and dosing schedule are established, the Disease Groups may be expanded in Part B with up to 12 additional patients to increase the safety experience and more extensively evaluate the PK and PD of XENP13676.

### Dosage and Mode of Administration

XENP13676 will be administered as an intravenous infusion at a constant rate over 2 hours every 7 days for 8 doses (2 cycles). Patients will be premedicated with dexamethasone 20 mg intravenously 1 hour prior to XENP13676 administration. XENP13676 drug product will be a liquid product supplied in single-use glass vials filled with 1 mL at a concentration of 5.0 mg/mL.

### Part A Escalation

Patients will be enrolled in parallel to both the NHL and CLL Disease Groups, and dose escalation in each Group will be essentially independent. Dose level increases will initially proceed according to an accelerated titration design. Once a priming dose has been determined (the lowest dose with occurrence of a single DLT), Part A will end and the study will enroll all patients to Part B from then on.

TABLE 3
Dose Escalation Cohorts

| Group | Cohort | Planned Dose | Subjects |
|---|---|---|---|
| Non-CLL B cell | 1N-A | 0.7 μg/kg | 1 (+2 + 3) |
| malignancies | 2N-A | 2.4 μg/kg | 1 (+2 + 3) |
| (Group NHL) | 3N-A | 7.5 μg/kg | 1 (+2 + 3) |
| | 4N-A | 20 μg/kg | 3 (+3) |
| | 5N-A | 45 μg/kg | 3 (+3) |
| | 6N-A | 80 μg/kg | 3 (+3) |
| | 7N-A | 125 μg/kg | 3 (+3) |
| | 8N-A | 170 μg/kg | 3 (+3) |
| CLL/SLL | 1C-A | 0.7 μg/kg | 1 (+2 + 3) |
| (Group CLL) | 2C-A | 2.4 μg/kg | 1 (+2 + 3) |
| | 3C-A | 7.5 μg/kg | 1 (+2 + 3) |
| | 4C-A | 20 μg/kg | 3 (+3) |
| | 5C-A | 45 μg/kg | 3 (+3) |
| | 6C-A | 80 μg/kg | 3 (+3) |
| | 7C-A | 125 μg/kg | 3 (+3) |
| | 8C-A | 170 μg/kg | 3 (+3) |

### Part B Escalation

In Part B, the Cycle 1 Day 1 dose (the priming dose) will be fixed at the level determined in Part A for each Disease Group. The second dose will be escalated and maintained at that level for subsequent doses. The dose to be examined in each cohort will be defined relative to the priming dose.

| | | Day 1 | | | | |
|---|---|---|---|---|---|---|
| | | (Priming | Day | Day | Day | |
| | Cohort | dose) | 8 | 15 | 22 | Patients |
| Part B | −1N-B or −1C-B | X | X | X + 1 | X + 1 | 3 (+3) |
| (Group | 1N-B or 1C-B | X | X + 1 | X + 1 | X + 1 | 3 (+3) |
| N or | 2N-B or 2C-B | X | X + 2 | X + 2 | X + 2 | 3 (+3) |
| Group | 3N-B or 3C-B | X | X + 3 | X + 3 | X + 3 | 3 (+3) |
| C) | 4N-B or 4C-B | X | X + 4 | X + 4 | X + 4 | 3 (+3) |
| | 5N-B or 5C-B | X | X + 5 | X + 5 | X + 5 | 3 (+3) |
| | 6N-B or 6C-B | X | X + 6 | X + 6 | X + 6 | 3 (+3) |
| | 7N-B or 7C-B | X | X + 7 | X + 7 | X + 7 | 3 (+3) |

| | | |
|---|---|---|
| Expansion-B | At MTD or RD cohort | Up to 12 |

for each
Disease
Group

Dose escalation will proceed by a standard 3+3 scheme and with the same dosing levels as in Part A, however the Cycle 1 Day 1 infusion will initially be the priming dose determined in Part A for that Disease Group (denoted as “X”). Dose escalation on each Part B cohort will be based on this starting point. For example, if the priming dose determined by Group NHL Part A is 20 μg/kg, the first infusion/priming dose in Cohort 1N-B will be 20 μg/kg and the second and subsequent infusions will be at 45 μg/kg (i.e. X+1).

A minimum of 3 patients will be enrolled in each cohort for each Disease Group. As in Part

A, no two patients within a cohort will start treatment with XENP13676 on the same day; the first patient will be dosed and observed for a minimum of 72 hours before study drug is administered to the remainder of the cohort.

The DLT observation period for the subsequent dosing escalation cohorts is Cycle 1 Day 8 through Cycle 2 Day 7. If all 3 patients tolerate a cohort without experiencing DLT (and the DERC agrees), enrollment will begin on the next higher cohort. If at any time during the 28-day observation period a DLT occurs, 3 additional patients will be added to the cohort. If there is an additional DLT among the 6 patients on the cohort, the previous dosing cohort will be expanded to 6 to establish a MTD and/or RD. If this occurs on cohort 1B, the next 3 patients will be enrolled on cohort-1B (de-escalation cohort). If there are no further DLTs among the 3 additional patients, another 3 patients will be added to the cohort. If there is an additional DLT, then the MTD/RD and schedule established in Part A for that Disease Group will be recommended for further study.

Toxicity rates for the priming dose will continued to be monitored during Part B by a probability boundary function applied from Cycle 1 Day 1 through Cycle 1 Day 7. Excess toxicity rates will trigger de-escalation of the priming dose.

Duration of treatment: Patients will receive two 4 week cycles of therapy (8 doses); a patient may continue on therapy past 2 cycles if, in the opinion of the investigator, he/she is deriving benefit and does not require additional non-study therapy.

### Example 3

### In Vitro Aantitumor Efficacy

The ability of XmAb13676 to recruit and redirect T cells to kill CD20-expressing target cells (RTCC) was examined. T cell-dependent cytotoxicity of XmAb13676 against CD20-positive Ramos cells was examined using purified PBMC or T cell-depleted PBMC as effector cells. In addition, T cell activation was examined by quantifying CD69 induction on both CD4+ and CD8+ T cells. XENP13245, an anti-RSV x anti-CD3 bispecific antibody, and XENP14045, an anti-CD123×anti-CD3 bispecific antibody, were included as negative controls.

XmAb13676 displayed robust and potent killing of Ramos cells when supplied with human PBMC as an effector population (data not shown). The negative control antibodies failed to induce any tumor cell killing, suggesting that the cytotoxicity mediated by XmAb13676 depends on its engagement of CD20 on the target cell population. However, when T cells were depleted from PBMC, XmAb13676 failed to induce killing. In a second experiment, XmAb13676-mediated killing of Ramos cells was demonstrated with purified T cells, demonstrating that T cells alone are sufficient to mediate XmAb13676′s cytotoxic activity.

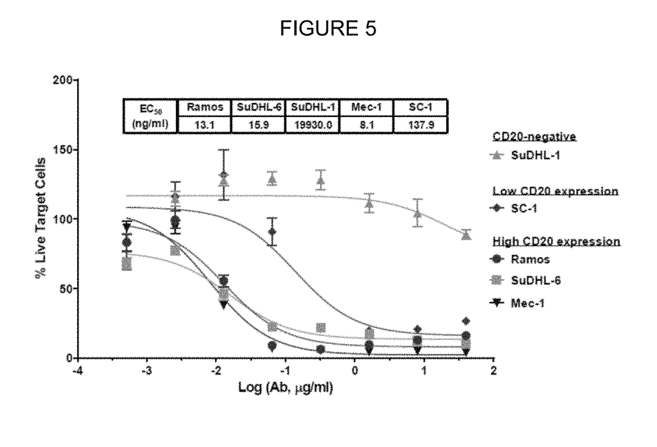

Using rituximab as the detection antibody, the CD20 expression profile on various human lymphoma-derived cell lines was examined by flow cytometry. Su-DHL-6 showed the highest CD20 antigen density, with Ramos and MEC-1 showing intermediate levels, and SC-1 showing lower levels. Su-DHL-1 was used as a CD20-negative control cell line. FIG. 5 shows RTCC activity against these five cell lines using purified T cells. XmAb13676 showed robust depletion of all CD20-positive target cell lines in the presence of purified T cells. Potency of killing was higher for cell lines with high or intermediate CD20 levels (Su-DHL-6, Ramos and MEC-1), and reduced approximately 10-fold for the lower expressing SC-1, with EC₅₀ values ranging from 8 to 138 ng/ml. No significant depletion was observed for the CD20-negative cell line (Su-DHL-1).

XmAb13676 also induced similar robust CD8⁺ (FIG. 6) and CD4⁺ (not shown) T cell activation in the presence of CD20-expressing target cells, again correlating with CD20 expression levels. In contrast, XmAb13676 failed to induce activation of CD4⁺ and CD8⁺ T cells or target cell killing in the presence of CD20-negative SuDHL-1 cells.

To assess whether XmAb13676-induced cytotoxicity is affected by donor-to-donor T cell variability, purified T cells from six healthy human donors were tested in RTCC assays using Ramos as target cells. XmAb13676 robustly depleted Ramos target cells in the presence of effector T cells from six different healthy donors (data not shown). The depletion potency was similar across all six donors, with EC₅₀ values between 5.3 and 14.3 ng/ml.

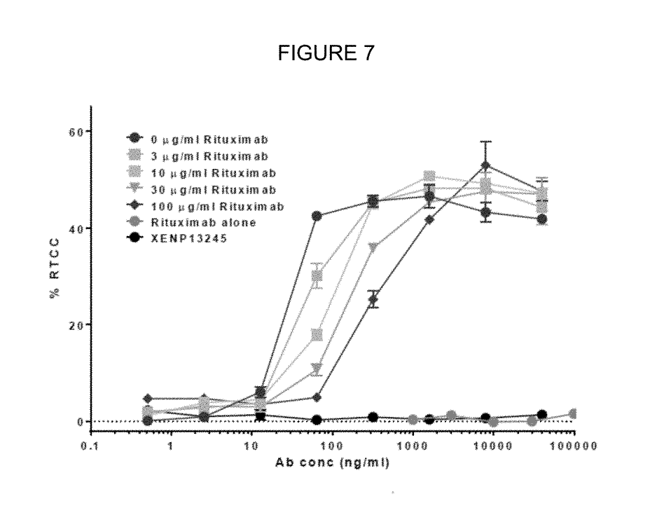

The anti-CD20 Fv domain of XmAb13676 is derived from murine antibody C2B8, the same antibody used in the chimeric antibody rituximab. Therefore, it might be possible that rituximab could interfere with the activity of XmAb13676 by competing for CD20 binding. To assess the effect of rituximab interference on XmAb13676-induced T cell- mediated cytotoxicity, its potency was evaluated in the presence of increasing amounts of rituximab. As shown in FIG. 7, XmAb13676 stimulated Jeko-1 target cell killing with an EC₅₀ of 24 ng/ml in the absence of rituximab. As rituximab was added at increasing concentrations of 3, 10, 30 and 100 μg/ml, the potency of XmAb13676 was correspondingly reduced (with EC₅₀ values increasing to 51, 93, 162 and 387 ng/ml). However, XmAb13676 retains RTCC activity even in the presence of a large excess concentration of rituximab. Moreover, although XmAb13676 became less potent in the presence of rituximab, it stimulated a similar extent of total T cell-mediated target killing efficacy. As expected, rituximab itself did not display any RTCC activity in this T cell-dependent assay.

A series of experiments was performed to assess whether XmAb13676 could induce T cell activity in cancer patient samples. T cell activation and killing were examined using patient-derived PBMC from CLL or follicular NHL (FL) patients. T cell activation and depletion of autologous CD20-expressing target B cells derived from normal PBMC samples were also examined as a benchmark. As a non-specific antigen control, XENP13245 (anti-RSV x anti-CD3 bsAb) was used.

Three each of CLL and normal donor PBMC samples were assessed for target cell (CD19+ CDS+ lymphocytes for CLL cancer cells and CD19+for normal B cells) and effector T cell (CD4+ and CD8+ cells) number. The number of T cells in CLL samples was significantly lower than in normal PBMC, resulting in very low effector to target (E:T) ratios.

CLL and normal PBMC samples were incubated for 24 or 48 hours with 10, 1, 0.1 μg/ml of XmAb13676 or 10 μg/ml of the control antibody XENP13245. After incubation, target cell counts were determined for CLL and normal PBMC samples. XmAb13676 induced robust B cell depletion in the normal PBMC at either 24 or 48 hours, resulting in a drop of several orders of magnitude in detectable B cells (not shown). This depletion activity appears to saturate at concentrations of 1 μg/ml or higher. However, there was no depletion of CD19⁺ CD5⁺ cells in the CLL samples, presumably due to the low number of effector T cells in these samples. The nonspecific control antibody XENP13245 (10 μg/ml) did not decrease target cell counts in either sample set.

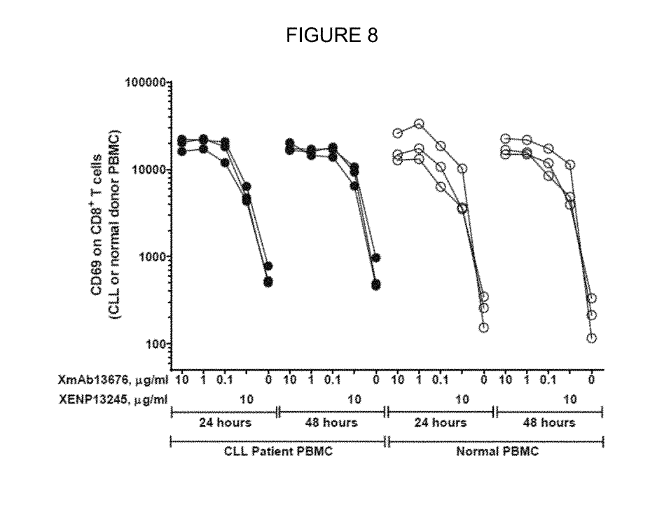

XmAb13676 engagement of CD3 on the effector T cells and CD20 on the target cells is expected to activate T cells, which can be measured by detecting surface activation markers such as CD25 and CD69. As shown in FIG. 8, CD8⁺ T cells were strongly activated by XmAb13676, as evidenced by the upregulation of CD69. Similar results were observed for CD25, and both CD69 and CD25 were also upregulated on CD4⁺ T cells (data not shown). Hence, despite the lack of CLL depletion, XmAb13676 mediates strong activation of autologous T cells in the CLL samples. Such activation may lead to proliferation in vivo, potentially overcoming the problem with low T cell counts.

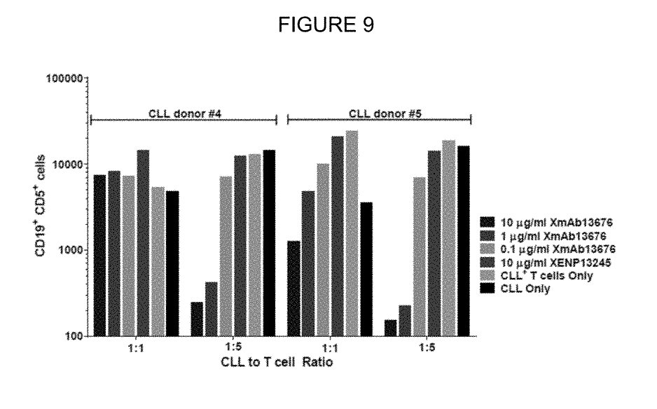

As the number of effector T cells in the CLL samples was low, two CLL samples were supplemented with T cells purified from normal PBMC to assess the sensitivity of the CLL cancer cells to the XmAb13676 bispecific antibody. The number of live PBMCs assayed from each CLL donor was 250,000 and 320,000 and purified T cells from a normal PBMC were added at equal number (1:1 ratio) or 5-fold access over the CLL cells (5:1 ratio of T cells to CLL cells) and incubated for 24 hours. FIG. 9 shows the number of CLL cancer cell counts in two CLL patient PBMC incubated with either XmAb13676 (at 0, 0.1, 1 or 10 μg/ml) or the negative control antibody XENP13245 (at 10 μg/ml). XmAb13676 induced very effective depletion of CD19+CD5+CLL cancer cells in both CLL patient PBMC samples at the 5:1 effector:target ratio, particularly at the highest concentration of 10 μg/ml. More modest depletion was observed at lower concentrations and at the lower 1:1 E:T ratio. In contrast, the negative control antibody XENP13245 (at 10 μg/ml) did not show any CLL cancer cell depletion at either 5:1 or 1:1 ratios. Therefore, CLL cells are sensitive to XmAb13676-induced T cell-mediated killing effects, in particular when sufficient T cells are present.

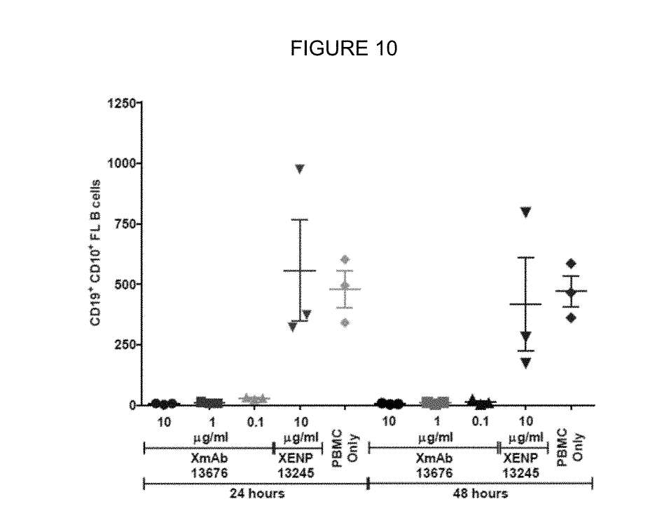

PBMC samples from three FL patients were also characterized for XmAb13676-mediated cytotoxicity. Although CD19+CD10+ FL cells were not high in number in these FL patient-derived PBMC samples, counts were sufficiently high to reveal XmAb13676-mediated cytotoxicity of this target population. CD19+CD10+ FL counts in control samples not exposed to XmAb13676 ranged from below 200 to almost 1000. In the presence of 0.1, 1.0, or 10 μg/ml concentrations of XmAb13676, these cells were nearly completely eliminated, particularly at the two higher concentrations. The nonspecific control antibody did not have any apparent impact on the FL cells. Notably, XmAb13676 also induced robust killing of the autologous healthy CD19+B cells present in the PBMC samples, with a similar extent of depletion and concentration dependence, as shown in FIG. 10. Finally, in a pattern consistent with the observed depletion of B cells, XmAb13676 strongly activated T cells in the FL samples, as evidenced by CD69 and CD25 upregulation on both CD4 and CD8 T cells.

### Example 4

### Antitumor Activity in a Mouse Lymphoma Mode

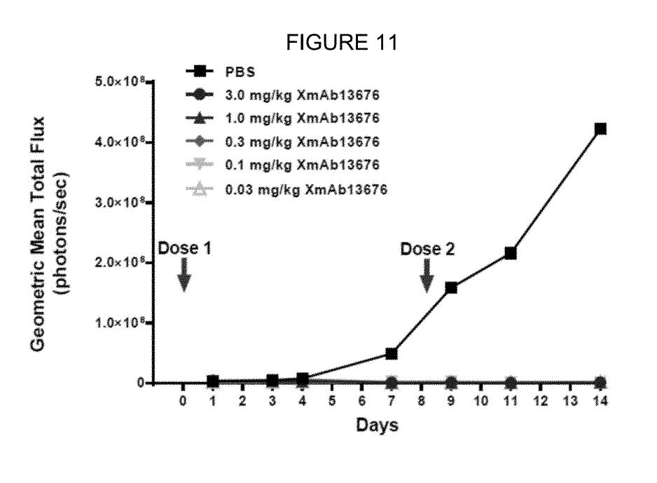

XmAb13676 does not cross-react with either mouse CD3 or CD20. Therefore, XmAb13676 was evaluated for its anti-tumor efficacy in NSG mice engrafted systemically with luciferase-transgenic human Raji cells (RajiTrS) as well as human PBMC. As shown in FIG. 11, In Vivo Imaging System (IVIS) analysis revealed that XmAb13676 prevented tumor growth at all doses tested, including 0.03 mg/kg, 0.1 mg/kg, 0.3 mg/kg, 1.0 mg/kg, 3.0 mg/kg. The data suggest that even lower doses would also be likely to have significant tumor prevention activity.

1. A method for treating lymphoma in a human subject, comprising:
administering to the human subject having lymphoma an intravenous dose of between about 0.1 μg/kg and about 200 μg/kg of a bispecific anti-CD20×anti-CD3 antibody once every 6-8 days for a time period sufficient to treat the lymphoma.

2. The method of claim 1, wherein the lymphoma is Non-Hodgkin lymphoma.

3. The method of claim 2, wherein the Non-Hodgkin lymphoma is B-cell NHL.

4. The method of claim 2, wherein the Non-Hodgkin lymphoma is selected from the group consisting of Burkitt's lymphoma (e.g., Endemic Burkitt's Lymphoma and Sporadic Burkitt's Lymphoma), Cutaneous B-Cell Lymphoma, Cutaneous Marginal Zone Lymphoma (MZL), Diffuse Large Cell Lymphoma (DLBCL), Diffuse Mixed Small and Large Cell Lymphoma, Diffuse Small Cleaved Cell, Diffuse Small Lymphocytic Lymphoma, Extranodal Marginal Zone B-cell lymphoma, follicular lymphoma, Follicular Small Cleaved Cell (Grade 1), Follicular Mixed Small Cleaved and Large Cell (Grade 2), Follicular Large Cell (Grade 3), Intravascular Large B-Cell Lymphoma, Intravascular Lymphomatosis, Large Cell Immunoblastic Lymphoma, Large Cell Lymphoma (LCL), Lymphoblastic Lymphoma, MALT Lymphoma, Mantle Cell Lymphoma (MCL), immunoblastic large cell lymphoma, precursor B-lymphoblastic lymphoma, mantle cell lymphoma, chronic lymphocytic leukemia (CLL)/small lymphocytic lymphoma (SLL), extranodal marginal zone B-cell lymphoma-mucosa-associated lymphoid tissue (MALT) lymphoma, Mediastinal Large B-Cell Lymphoma, nodal marginal zone B-cell lymphoma, splenic marginal zone B-cell lymphoma, primary mediastinal B-cell lymphoma, lymphoplasmocytic lymphoma, hairy cell leukemia, Waldenstrom's Macroglobulinemia, and primary central nervous system (CNS) lymphoma.

5. The method of claim 2, wherein the Non-Hodgkin lymphoma is chronic lymphocytic leukemia (CLL)/small lymphocytic lymphoma (SLL).

6. The method of a preceding claim 1, wherein the intravenous dose is:
between about 0.6 μg/kg and about 0.8 μg/kg; or
between about 2.3 μg/kg and about 2.5 μg/kg; or
between about 6.5 μg/kg and about 8.5 μg/kg; or
between about 18 μg/kg and about 22 μg/kg; or
between about 40 μg/kg and about 50 μg/kg; or
between about 75 μg/kg and about 85 μg/kg; or
between about 120 μg/kg and about 130 μg/kg; or
between about 165 μg/kg and about 175 μg/kg.

7. The method of claim 1, wherein the intravenous dose is administered to the human subject between about 1 hour and about 3 hours.

8. The method of claim 1, wherein the time period sufficient to treat the lymphoma is between about 3 weeks and 9 weeks.

9. The method of claim 1, wherein the bispecific anti-CD20×anti-CD3 antibody comprises: a first monomer comprising SEQ ID NO: 1, a second monomer comprising SEQ ID NO: 2, and a light chain comprising SEQ ID NO: 3 .

10. The method of claim 1, further comprising, prior to the administering of the bispecific anti-CD20×anti-CD3 antibody, administering a steroid to the human subject.

11. The method of claim 1, further comprising, prior to the administering of the bispecific anti-CD20×anti-CD3 antibody, assessing the weight of the human subject.

12. A method for treating a CD20-expressing cancer in a human subject, comprising:
administering to the human subject having the CD20-expressing cancer an intravenous dose of between about 0.45 μg/kg and about 110 μg/kg of a bispecific anti-CD20×anti-CD3 antibody monthly for a time period sufficient to treat the CD20-expressing cancer.

13. A method for treating a CD20-expressing cancer in a human subject, comprising:
administering to the human subject having the CD20-expressing cancer an intravenous dose of between about 0.45 μg/kg and about 110 μg/kg of a bispecific anti-CD20×anti-CD3 antibody every other week for a time period sufficient to treat the CD20-expressing cancer.

14. The method of claim 12, wherein the intravenous dose is between about 28 μg/kg and about 80 μg/kg

15. The method of claim 12, wherein the bispecific anti-CD20×anti-CD3 antibody comprises: a first monomer comprising SEQ ID NO: 1, a second monomer comprising SEQ ID NO: 2, and a light chain comprising SEQ ID NO: 3.

16. The method of claim 12, wherein the CD20-expressing cancer is a lymphoma.

17. The method of claim 16, wherein the lymphoma is a Non-Hodgkin lymphoma.

18. The method of claim 17, wherein the Non-Hodgkin lymphoma is B-cell NHL.

19. The method of claim 17, wherein the Non-Hodgkin lymphoma is selected from the group consisting of Burkitt's lymphoma (e.g., Endemic Burkitt's Lymphoma and Sporadic Burkitt's Lymphoma), Cutaneous B-Cell Lymphoma, Cutaneous Marginal Zone Lymphoma (MZL), Diffuse Large Cell Lymphoma (DLBCL), Diffuse Mixed Small and Large Cell Lymphoma, Diffuse Small Cleaved Cell, Diffuse Small Lymphocytic Lymphoma, Extranodal Marginal Zone B-cell lymphoma, follicular lymphoma, Follicular Small Cleaved Cell (Grade 1), Follicular Mixed Small Cleaved and Large Cell (Grade 2), Follicular Large Cell (Grade 3), Intravascular Large B-Cell Lymphoma, Intravascular Lymphomatosis, Large Cell Immunoblastic Lymphoma, Large Cell Lymphoma (LCL), Lymphoblastic Lymphoma, MALT Lymphoma, Mantle Cell Lymphoma (MCL), immunoblastic large cell lymphoma, precursor B-lymphoblastic lymphoma, mantle cell lymphoma, chronic lymphocytic leukemia (CLL)/small lymphocytic lymphoma (SLL), extranodal marginal zone B-cell lymphoma-mucosa-associated lymphoid tissue (MALT) lymphoma, Mediastinal Large B-Cell Lymphoma, nodal marginal zone B-cell lymphoma, splenic marginal zone B-cell lymphoma, primary mediastinal B-cell lymphoma, lymphoplasmocytic lymphoma, hairy cell leukemia, Waldenstrom's Macroglobulinemia, and primary central nervous system (CNS) lymphoma.

20. The method of claim 17, wherein the Non-Hodgkin lymphoma is chronic lymphocytic leukemia (CLL)/small lymphocytic lymphoma (SLL).

21. The method of claim 20, further comprising administering to the human subject another agent selected from an alkylating agent such as bendamustine hydrochloride (e.g., Treanda), chlorambucil (e.g., Leukeran, Ambochlorin, Amboclorin, Linfolizin), cyclophosphamide (e.g., Cytoxan, Clafen, Neosar); a purine analog such as fludarabine phosphate (e.g., Fludara), cladribine (e.g., Leustatin, 2-CdA), pentostatin (Nipent®); an Bcl2 inhibitor such as ABT-737, venetoclax (e.g., Venclexta); a kinase inhibitor such as ibrutinib (e.g., Imbruvica), venetoclax, idelalisib (e.g., Zydelig); an anti-CD52 Ab such as alemtuzumab (Campath®); a corticosteroid such as prednisone, methylprednisolone, or dexamethasone; or CVP (a combination of cyclophosphamide, vincristine, and prednisone), CHOP (a combination of cyclophosphamide, hydroxydaunorubicin, Oncovin® (vincristine), and prednisone) with or without etoposide (e.g., VP-16), a combination of cyclophosphamide and pentostatin, a combination of chlorambucil and prednisone, a combination of fludarabine and cyclophosphamide, or another agent such as mechlorethamine hydrochloride (e.g. Mustargen), doxorubicin (Adriamycin®), methotrexate, oxaliplatin, or cytarabine (ara-C).

22. The method of claim 1 further comprising administering to said subject another therapy.

23. The method of claim 22, wherein said another therapy is a chemotherapy.

24. The method of claim 23, wherein said chemotherapy is selected from the group consisting of : a anthracycline (e.g., idarubicin, daunorubicin, doxorubicin (e.g., liposomal doxorubicin)), a anthracenedione derivative (e.g., mitoxantrone), a vinca alkaloid (e.g., vinblastine, vincristine, vindesine, vinorelbine), an alkylating agent (e.g., cyclophosphamide, deacarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, cytarabine, pyrimidine analogs, purine analogs and adenosine deaminase inhibitors (e.g., fludarabine)), an mTOR inhibitor, a proteasome inhibitor (e.g., aclacinomycin A, gliotoxin or bortezomib), an immunomodulator such as thalidomide or a thalidomide derivative (e.g., lenalidomide).

25. The method of claim 22, wherein said another therapy is a therapy that ameliorates side effects.

26. The method of claim 25, wherein said another therapy is selected from the group consisting of: a steroid (e.g., corticosteroid, e.g., methylprednisolone, hydrocortisone), an inhibitor of TNFα, inhibitor of IL-1R, and an inhibitor of IL-6.

27. The method of claim 26, wherein said another therapy is a combination of a corticosterioid (e.g., methylprednisolone, hydrocortisone) and Benadryl and Tylenol, wherein said corticosterioid, Benadryl and Tylenol are administered to said subject prior to the administration of said anti-CD20×anti-CD3 antibody.


SEQUENCE LISTING (21 sequences)

SEQ ID NO: 1 - protein, 450 aa, Artificial Sequence
  XENP13676 Anti-CD20 x Anti-CD3 Fab-scFv-Fc Heavy Chain 1
QVQLVQSGAEVKKPGASVKVSCKASGYTFTSYNMHWVRQAPGQGLEWMGAIYPGNGDTSYNQKFQGRVTITADKSISTAYMELSSLRSEDTAVYYCARSTYYGGDWYFNVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 2 - protein, 485 aa, Artificial Sequence
  XENP13676 Anti-CD20 x Anti-CD3 Fab-scFv-Fc Heavy Chain 2
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 213 aa, Artificial Sequence
  XENP13676 Anti-CD20 x Anti-CD3 Fab-scFv-Fc Light Chain
QIVLTQSPSSLSASVGDRVTITCRASSSVSYIHWFQQKPGKSPKPLIYATSNLASGVPVRFSGSGSGTDYTLTISSLQPEDFATYYCQQWTSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 450 aa, Artificial Sequence
  XENP13677 Anti-CD20 x Anti-CD3 Fab-scFv-Fc Heavy Chain 1
QVQLVQSGAEVKKPGASVKVSCKASGYTFTSYNMHWVRQAPGQRLEWMGAIYPGNGATSYSQKFQGRVTITADTSASTAYMELSSLRSEDTAVYYCARSYYMGGDWYFDVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 5 - protein, 485 aa, Artificial Sequence; identical to SEQ ID NO: 2
  XENP13677 Anti-CD20 x Anti-CD3 Fab-scFv-Fc Heavy Chain 2

SEQ ID NO: 6 - protein, 213 aa, Artificial Sequence
  XENP13677 Anti-CD20 x Anti-CD3 Fab-scFv-Fc Light Chain
QIVLTQSPSSLSASVGDRVTITCRASWSVSYIHWFQQKPGKSPKPLIYATSNLASGVPVRFSGSGSGTDYTLTISSLQPEDFATYYCQQWTHNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 450 aa, Artificial Sequence; identical to SEQ ID NO: 4
  XENP14388 Anti-CD20 x Anti-CD3 Fab-scFv-Fc Heavy Chain 1

SEQ ID NO: 8 - protein, 485 aa, Artificial Sequence
  XENP14388 Anti-CD20 x Anti-CD3 Fab-scFv-Fc Heavy Chain 2
EVQLVESGGGLVQPGGSLRLSCAASGFTFSTYAMSWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 213 aa, Artificial Sequence; identical to SEQ ID NO: 6
  XENP14388 Anti-CD20 x Anti-CD3 Fab-scFv-Fc Light Chain

SEQ ID NO: 10 - protein, 450 aa, Artificial Sequence; identical to SEQ ID NO: 4
  XENP14389 Anti-CD20 x Anti-CD3 Fab-scFv-Fc Heavy Chain 1

SEQ ID NO: 11 - protein, 485 aa, Artificial Sequence
  XENP14389 Anti-CD20 x Anti-CD3 Fab-scFv-Fc Heavy Chain 2
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 213 aa, Artificial Sequence; identical to SEQ ID NO: 6
  XENP14389 Anti-CD20 x Anti-CD3 Fab-scFv-Fc Light Chain

SEQ ID NO: 13 - protein, 450 aa, Artificial Sequence; identical to SEQ ID NO: 4
  XENP14390 Anti-CD20 x Anti-CD3 Fab-scFv-Fc Heavy Chain 1

SEQ ID NO: 14 - protein, 485 aa, Artificial Sequence
  XENP14390 Anti-CD20 x Anti-CD3 Fab-scFv-Fc Heavy Chain 2
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 213 aa, Artificial Sequence; identical to SEQ ID NO: 6
  XENP14390 Anti-CD20 x Anti-CD3 Fab-scFv-Fc Light Chain

SEQ ID NO: 16 - protein, 450 aa, Artificial Sequence; identical to SEQ ID NO: 4
  XENP14392 Anti-CD20 x Anti-CD3 Fab-scFv-Fc Heavy Chain 1

SEQ ID NO: 17 - protein, 485 aa, Artificial Sequence
  XENP14392 Anti-CD20 x Anti-CD3 Fab-scFv-Fc Heavy Chain 2
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 213 aa, Artificial Sequence; identical to SEQ ID NO: 6
  XENP14392 Anti-CD20 x Anti-CD3 Fab-scFv-Fc Light Chain

SEQ ID NO: 19 - protein, 450 aa, Artificial Sequence; identical to SEQ ID NO: 4
  XENP14393 Anti-CD20 x Anti-CD3 Fab-scFv-Fc Heavy Chain 1

SEQ ID NO: 20 - protein, 485 aa, Artificial Sequence
  XENP14393 Anti-CD20 x Anti-CD3 Fab-scFv-Fc Heavy Chain 2
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDPYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 213 aa, Artificial Sequence; identical to SEQ ID NO: 6
  XENP14393 Anti-CD20 x Anti-CD3 Fab-scFv-Fc Light Chain